(19) United States
(12) Patent Application Publication
(10) Pub. No.: US 2018/0214563 A1
(43) Pub. Date: Aug. 2, 2018

(54) IMMUNOSTIMULATORY NANOCARRIER

(71) Applicant: University of Pittsburgh - Of the Commonwealth System of Higher Education, PITTSBURGH, PA (US)

(72) Inventors: Song Li, Wexford, PA (US); Yichao Chen, Pittsburgh, PA (US); Yixian Huang, Pittsburgh, PA (US)

(21) Appl. No.: 15/748,469

(22) PCT Filed: Jul. 28, 2016
(86) PCT No.: PCT/US2016/044384

§ 371 Date: Jan. 29, 2018

Related U.S. Application Data
(60) Provisional application No. 62/199,455, filed on Jul. 31, 2015.

(51) Int. Cl. A61K 47/60; A61K 47/69; A61K 45/06

(52) U.S. Cl. CPC A61K 47/60; A61K 47/6907; A61K 31/704; A61K 2300/00; A61K 45/06

(57) ABSTRACT

A formulation includes a carrier agent formed by conjugating an immunotherapy agent with a hydrophilic compound. The carrier agent further includes an interactive domain comprising at least one interactive moiety which interacts with a therapeutic agent.

### CROSS-REFERENCE TO RELATED APPLICATIONS

This application claims benefit of U.S. Provisional Patent Application Ser. No. 62/199,455, filed Jul. 31, 2015, the disclosure of which is incorporated herein by reference.

### GOVERNMENTAL INTEREST

This invention was made with government support under grant no. RO1CA174305 and R01GM102989 awarded by the National Institutes of Health. The government has certain rights in this invention.

### BACKGROUND

The following information is provided to assist the reader in understanding technologies disclosed below and the environment in which such technologies may typically be used. The terms used herein are not intended to be limited to any particular narrow interpretation unless clearly stated otherwise in this document. References set forth herein may facilitate understanding of the technologies or the background thereof. The disclosure of all references cited herein are incorporated by reference.

Chemotherapy remains a mainstay treatment for various types of cancers. It is generally regarded that chemotherapeutics work through cytostatic and/or cytotoxic effects. Accumulating evidence suggests that chemotherapy-elicited immune responses also contribute significantly to the overall antitumor activity. Chemotherapeutic agents can modify the propensity of malignant cells to elicit an immune response and/or directly exert immunostimulatory effects. For example, significant interferon gamma (IFN-γ) response was found in 4T1.2 cell line tumor tissue following treatment of tumor-bearing mice with TAXOL® (paclitaxel). However, the effectiveness of chemotherapy-elicited immune response as well as other immunotherapies is limited by various negative feedback mechanisms that are upregulated during the cancer treatment. For example, programmed cell death protein 1 (PD1) is a key immune-checkpoint receptor expressed on activated T-cells, which negatively regulates immune response thorough binding to its ligand, PD-L1. By utilizing this pathway, cancer cells can protect themselves from tumor-specific T-cells. A recent study has shown that, in patient with papillomavirus (HPV)-related oropharyngeal cancer, the PD-1 expression levels on CD4⁺ T cells were increased nearly 2.5-fold at 3 weeks after completion of chemoradiation. On the other hand, the tumor cells can upregulate PD-L1 to decrease cytotoxic lymphocytes attack, and this upregulation is possibly a consequence of pro-inflammatory cytokine (e.g., IFN-γ) production by tumor infiltrating immune cells after cancer therapy. Therapeutics that are targeted at PD-1, such as PD-1 monoclonal antibodies, are currently being tested as a new strategy to improve the treatment of cancers.

Indoleamine-pyrrole 2,3-dioxygenase (IDO) is another checkpoint protein involved in generating the immunosuppressive microenvironment that supports tumor cells growth. IDO is an enzyme catalyzing the degradation of essential amino acid tryptophan. IDO overexpressed in some cancer cells exerts depletion of tryptophan and accumulation of its metabolites, resulting in cell cycle arrest and death of effector T cells and direct activation the regulatory T cells.

Immunotherapy strategies (such as those, that are targeted at PD-1 and IDO) represent an attractive approach for the treatment of cancer, particularly in combination with chemotherapy. However, many immunotherapy agents are poorly water soluble and their in vivo applications require complicated protocols. Moreover, co-delivery of immunotherapy agents and chemotherapeutic agents to tumors remains a challenge as a result of their different physical and pharmacokinetic profiles.

### SUMMARY

In one aspect, a formulation includes a carrier agent formed by conjugating an immunotherapy agent with a hydrophilic compound. The carrier agent further includes an interactive domain comprising at least one interactive moiety which interacts with a co-delivered therapeutic agent. In a number of embodiments, the immunotherapy agent is conjugated to the hydrophilic compound via a linkage which is labile in vivo. The at least one interactive group may interact with a therapeutic agent such as a chemotherapy agent (for example, have an affinity therefor). The immunotherapy agent may, for example, affect programmed cell death protein, indoleamine-pyrrole 2,3-dioxygenase, cytotoxic T-lymphocyte antigen 4(CTLA-4), PD-L1, PD-L2, lymphocyte activation gene 3(LAG3), or B7 homolog3(B7-H3). In a number of embodiments, the immunotherapy agent is NLG919 or derivative thereof. In a number of embodiments, the immunotherapeutic agent is a polymer formed from immunetherapeutically active monomers.

The interactive domain may, for example, include at least one of a fluorenylmethyloxycarbonyl group, a carbobenzyloxy group, an isobutoxycarbamate group, a naphthylacetyl group, a carbazole group, a quinolone group, an isoquinolone group, or a group which is a residue of a molecule selected from the group of the compound, a portion of the compound, (9H-fluoren-9-yl)methanamine, (9H-fluoren-9-yl)methanol, 9H-fluoren-9-amine, naphthalene, 1,1′-bi-2-naphthol (BINOL), camptothecin, a camptothecin analog, pemetrexed, docetaxel, paclitaxel, epirubicin, doxorubicin, vinblastine, vindesine, etoposide, hydroxycamptothecin, irinotecan, mitoxantrone, tamoxifen, tretinoin, Vitamin A, Vitamin E, Vitamin K, Vitamin D, curcumin, imatinib, gefitinib, erlotinib, sorafenib, and bortezomib, or a derivative thereof. In a number of embodiments, the compound interactive domain includes at least one fluorenylmethyloxycarbonyl group or a derivative thereof.

The hydrophilic compound may, for example, include at least one hydrophilic oligomer or at least one hydrophilic polymer. The hydrophilic oligomer or the hydrophilic polymer may, for example, be a polyalkylene oxide, a polyvinylalcohol, a polyacrylic acid, a polyacrylamide, a polyoxazoline, or a polypeptide. In a number of embodiments, the polyalkylene oxide is a polyethylene glycol.

As described above, the at least one interactive group may, for example, have an affinity for the co-delivered therapeutic agent. The at least interactive group may, for example, interacts with the therapeutic agent via π-π stacking, hydrophobic interaction or hydrogen-bonding.

In a number of embodiments, the carrier agent provides a loading capacity for the therapeutic agent of at least 10%, at least 20%, at least 30% or greater.

As described above, the therapeutic agent, wherein the therapeutic agent is a chemotherapeutic agent. The chemotherapeutic agent may, for example be paclitaxel, doxorubicin, docetaxel, gefitinib, imatinib, dasatinib, curcumin, camptothecin, etoposide, edelfosine, vincristine, temsirolimus, carmustine, or a chemotherapeutically active derivative thereof.

In another aspect, a method of forming a formulation includes forming a carrier agent by conjugating an immunotherapy agent with a hydrophilic compound, the carrier agent further includes an interactive domain comprising at least one interactive moiety which interacts with a co-delivered therapeutic agent.

In a further aspect, a method of treating a patient with a therapeutic agent includes delivering to the patient a formulation, wherein the formulation includes the therapeutic agent and a carrier agent formed by conjugating an immunotherapy agent with a hydrophilic compound. The carrier agent further includes an interactive domain comprising at least one interactive moiety which interacts with a co-delivered therapeutic agent. The interactive domain may, for example, be positioned between a residue of the therapeutic agent and a residue of the hydrophilic compound in the carrier agent. In a number of embodiments, the immunotherapy agent is conjugated to the hydrophilic compound via a linkage which is labile in vivo.

The present systems, methods and compositions, along with the attributes and attendant advantages thereof, will best be appreciated and understood in view of the following detailed description taken in conjunction with the accompanying drawings.

### BRIEF DESCRIPTION OF THE DRAWINGS

FIG. 1A illustrates representative embodiments of synthesis schemes of two representative PEG2K-Fmoc-NLG conjugates, one with a relatively labile ester linkage (PEG2K-Fmoc-NLG(L)) and the other one with a relatively stable amide linkage (PEG2K-Fmoc-NLG(S)).

FIG. 1B illustrates PEG₂ₖ-Fmoc-NLG inhibited IDO enzyme activity in vitro, wherein HeLa cells were treated with IFN-γ together with free NLG919 or PEG-NLG conjugate and kynurenine in supernatants was measured 2 days later. *P<0.05 (vs PEG₂ₖ-Fmoc-NLG(L), N=3), ^{#}P<0.05 (vs PEG₂ₖ-Fmoc-NLG(S), N=3).

FIG. 1C illustrates IDO1 inhibition reversed T-cell suppression mediated by IDO-expressing mouse pancreatic cancer cells (Panc02), wherein Panc02 cells and splenocytes were mixed and treated with IL-2, anti-CD3 antibody, IFN-γ together with NLG919 or PEG-NLG conjugate for 3 days, and wherein T cell proliferation was examined by FACS (representative data of 3 independent experiments are presented. *P<0.05

FIG. 1D illustrates PEG₂ₖ-Fmoc-NLG(L) treatment decreased kynurenine concentrations in plasma and tumors, wherein BALB/c mice bearing s.c. 4T1.2 tumors of ˜100 mm³ received PBS or PEG₂ₖ-Fmoc-NLG(L) i.v. once every 3 days for 5 times at a dose of 25 mg NLG919/kg, and wherein kynurenine/tryptophan ratios in plasma and tumors were determined by LC/MS one day following the last injection. Data are means±s.e.m. of 3 experiments. *P<0.05, **P<0.01.

FIG. 1E(i) illustrates IDOL inhibition by PEG₂ₖ-Fmoc-NLG(L) increased CD4⁺ and CD8⁺ T cells, and decreased T_{reg} cells in tumors in mice, wherein the upper panel shows gating of CD8⁺ and CD4⁺ T cells (marked with boxes) as a percentage of CD45⁺ lymphocytes, and the lower panel shows gating of T_{reg} (CD4⁺ FoxP3⁺) cells (marked with boxes) as a percentage of CD4⁺ lymphocytes.

FIG. 1E(ii) illustrates IDO1 inhibition by PEG₂ₖ-Fmoc-NLG(L) increased CD4⁺ and CD8⁺ T cells, and decreased T_{reg} cells in tumors in mice, wherein the upper panel shows relative number of intratumoral CD8⁺ T cells following different treatments, and the lower panel shows relative number of T_{reg} cells and CD8⁺ Teff/T_{reg} in tumor tissues. Data represent means±s.e.m. (**P<0.01, N=5).

FIG. 1F illustrates tumor volume as a function of time showing that PEG₂ₖ-Fmoc-NLG maintained the tumor inhibitory effect in mice bearing tumors of ˜50 mm³ which received different treatments as indicated by black arrows. *P<0.05; **P<0.01 (vs control, N=5), ^{#P<}0.05 (vs PEG₂ₖ-Fmoc-NLG(S), N=5).

FIG. 1G illustrates tumor volume as a function of time showing that lymphocyte activities were required for the in vivo activity of PEG₂ₖ-Fmoc-NLG(L) micelles in female BALB/c-nu/nu mice bearing 4T1.2 tumor of ˜50 mm³ which were treated in a manner similar to that described in connection with FIG. 1F.

FIG. 1H illustrates tumor volume as a function of time showing enhanced in vivo antitumor activity of PEG₂ₖ-Fmoc-NLG(L) compared to oral delivery of NLG (^{#}P<0.05, N=5) or NLG formulated in PEG₅ₖ-(Fmoc-Boc)₂ micelles (^{&}P<0.05, N=5), *P<0.05 (vs control, N=5).

FIG. 2A illustrates size distribution and morphology of drug-free and PTX-loaded PEG₂ₖ-Fmoc-NLG(L) micelles (Carrier: drug, 2.5:1, m/m) examined by DLS and TEM, respectively, wherein drug concentration in micelles was kept at 1 mg/mL and blank micelle concentration was 20 mg/mL.

FIG. 2B illustrates measurement of critical micelle concentration (CMC) of PEG₂ₖ-Fmoc-NLG(L) micelles.

FIG. 2C illustrates sizes and drug-loading capacity (DLC) of various drug-loaded PEG₂ₖ-Fmoc-NLG(L) micelles

FIG. 2D illustrates PTX release kinetics of PTX/PEG₂ₖ-Fmoc-NLG(L) examined via a dialysis method, wherein PTX concentrations were kept at 1 mg/mL in both PTX/PEG₂ₖ-Fmoc-NLG(L) and Taxol, and PTX concentration was analyzed at 0, 1, 2, 4, 8, 24 and 48 h by HPLC.

FIG. 2E illustrates cytotoxicity of PEG₂ₖ-Fmoc-NLG(L) alone, free PTX, and micellar PTX against a mouse breast cancer cell line (4T1.2 ) and a human prostate cancer cell line (PC3), wherein cells were treated for 72 h and cytotoxicity was determined by MTT assay. *P<0.05 (PTX/PEG₂ₖ-Fmoc-NLG(L) vs PTX), N=3.

FIG. 2F illustrates cytotoxicity of PEG₂ₖ-Fmoc-NLG(L) alone, free DOX, and micellar DOX against a mouse breast cancer cell line (4T1.2 ) and a human prostate cancer cell line (PC3).

FIG. 2G illustrates IC50 of PTX or DOX in different formulations.

FIG. 3A illustrates a study of the kinetics of NLG in blood in 4T1.2 tumor-bearing mice following i.v. administration of PEG₂ₖ-Fmoc-NLG(L) in comparison to NLG-loaded PEG₅ₖ-(Fmoc-Boc)₂ micelles (25 mg NLG/kg).

FIG. 3B illustrates a study of the kinetics of NLG in a tumor in 4T1.2 tumor-bearing mice following i.v. administration of PEG₂ₖ-Fmoc-NLG(L) in comparison to NLG-loaded PEG₅ₖ-(Fmoc-Boc)₂ micelles (25 mg NLG/kg).

FIG. 3C illustrates tissue distribution of NLG in 4T1.2 tumor-bearing BALB/c mice following i.v. administration of PEG₂ₖ-Fmoc-NLG(L) micelles at a NLG dose of 25 mg/kg.

FIG. 3D illustrates tissue distribution of NLG in 4T1.2 tumor-bearing BALB/c mice following i.v. administration of NLG-loaded PEG₅ₖ-(Fmoc-Boc)₂ micelles at a NLG dose of 25 mg/kg.

FIG. 3E illustrates blood kinetics of PTX in BALB/c mice following i.v. administration of Taxol or PTX/PEG₂ₖ-Fmoc-NLG(L) mixed micelles at a dose of 10 mg PTX/kg.

FIG. 3F illustrates pharmacokinetic variables of Taxol and PTX/PEG₂ₖ-Fmoc-NLG(L) mixed micelles.

FIG. 3G illustrates tissue distribution of PTX in 4T1.2 tumor-bearing BALB/c mice 24 h following i.v. administration of Taxol or PTX/PEG₂ₖ-Fmoc-NLG(L) mixed micelles at a PTX dose of 10 mg/kg. *P<0.05 (N=5).

FIG. 3H illustrates tissue distributions of PTX at various time points with i.v. administration of Taxol.

FIG. 3I illustrates tissue distributions of PTX/PEG₂ₖ-Fmoc-NLG(L) mixed micelles (i) (10 mg PTX/kg).

FIG. 4A illustrates in vivo antitumor activity of various PTX formulations in 4T1.2 tumor model (PTX dose was 10 mg/kg)m wherein tumor sizes were plotted as relative tumor volume. **P<0.01 (all treatment groups vs control group), ^{#}P<0.05 (PTX/PEG₂ₖ-Fmoc-NLG(L) vs Taxol), ^{&}P<0.05 (PTX/PEG₂ₖ-Fmoc-NLG(L) vs PTX/PEG₂ₖ-Fmoc-NLG(S)).

FIG. 4B illustrates a dose-escalation study on the antitumor activity of PTX-loaded PEG₂ₖ-Fmoc-NLG(L) micelles. PTX dose was 5, 10, and 20 mg/kg, respectively. **P<0.01 (all treatment groups vs control), ^{#}P<0.05 (20 mg PTX/kg vs 5 mg PTX/kg).

FIG. 4C illustrates antitumor activity of PTX/PEG₂ₖ-Fmoc-NLG(L) in a 4T1.2 tumor model in comparison to a combination of oral NLG with i.v. Abraxane, PEG₂ₖ-Fmoc-NLG(L) plus Abraxane or PEG₅ₖ-(Fmoc-Boc)₂ ) micelles co-loaded with PTX and NLG. *P<0.01 (all treatment groups vs control), ^{#}P<0.05 (PTX/PEG₂ₖ-Fmoc-NLG(L) or PEG₂ₖ-Fmoc-NLG(L)+Abraxane vs Oral NLG+Abraxane), ^{&}P<0.05 (PTX/PEG₂ₖ-Fmoc-NLG(L) or PEG₂ₖ-Fmoc-NLG(L)+Abraxane vs (PTX+NLG)/PEG₅ₖ-(Fmoc-Boc)₂), ^{┐}P<0.05 (PTX/PEG₂ₖ-Fmoc-NLG(L) vs PEG₂ₖ-Fmoc-NLG(L)+Abraxane), N=5.

FIG. 4D illustrates antitumor activity of PTX/PEG₂ₖ-Fmoc-NLG(L) in a murine melanoma (B16) model. PTX dose was 10 mg/kg. **P<0.01 (all treatment groups vs control), ^{#}P<0.05 (PTX/PEG₂ₖ-Fmoc-NLG(L) vs Taxol), N=5.

FIG. 5A illustrates T cell infiltration in mouse tumors treated with Taxol, PEG₂ₖ-Fmoc-NLG(L) or PTX/PEG₂ₖ-Fmoc-NLG(L) at a PTX dosage of 10 mg/kg, wherein the relative abundance of CD4⁺, CD8⁺ T cells in tumor tissues were detected by flow cytometer.

FIG. 5B illustrates T cell infiltration in mouse tumors treated with Taxol, PEG₂ₖ-Fmoc-NLG(L) or PTX/PEG₂ₖ-Fmoc-NLG(L) at a PTX dosage of 10 mg/kg, wherein the relative abundance of IFN-γ positive intratumoral CD4⁺ T cells in tumor tissues were detected by flow cytometer.

FIG. 5C illustrates T cell infiltration in mouse tumors treated with Taxol, PEG₂ₖ-Fmoc-NLG(L) or PTX/PEG₂ₖ-Fmoc-NLG(L) at a PTX dosage of 10 mg/kg, wherein the relative abundance of IFN-γ positive intratumoral CD8⁺ T cells in tumor tissues were detected by flow cytometer.

FIG. 5D illustrates T cell infiltration in mouse tumors treated with Taxol, PEG₂ₖ-Fmoc-NLG(L) or PTX/PEG₂ₖ-Fmoc-NLG(L) at a PTX dosage of 10 mg/kg, wherein the relative abundance of granzyme B-positive CD8⁺ T cells in tumor tissues were detected by flow cytometer.

FIG. 5E illustrates flow cytometry gating and histogram analysis of FoxP3⁺ T regulatory cells in mouse tumors.

FIG. 5F illustrates tumor-associated macrophages (TAMs) in mouse tumors. Flow cytometry gating of the M1-type (CD11b⁺/F4/80⁺/CD206⁻) and the M2-type (CD11b⁺/F4/80⁺/CD206⁺) and histogram of M1/M2 ratios.

FIG. 5G illustrates flow cytometry gating and histograms analysis of CD11b⁺/Gr-1⁺ MDSC cells in mouse tumors, wherein double positive cells contain two populations, including Gr-1^{high}CD11b⁺ granulocytic (G-MDSC) and Gr-1^{int}CD11b⁺ monocytic (M-MDSC) MDSC subsets. The Bars represent means±s.e.m. (*p<0.05, **p<0.01, N=3).

FIG. 6 illustrates representative examples of the chemical structure of a number of IDO inhibitors, PD1-PDL1 inhibitors, and TDO inhibitors suitable for user herein as conjugated immunotherapy agents.

FIG. 7A illustrates NLG-919 and a number of polymerizable NLG-919 analogs or derivative monomers.

FIG. 7B illustrates two representative methods for polymerization of the monomers of FIG. 6A.

FIG. 8A illustrates a representative example of a PD-L1 inhibiting immunotherapy agent suitable for use herein and a number of polymerizable analogs/monomers thereof.

FIG. 8B illustrates representative ATRP and RAFT polymerizations suitable for use with the analogs/monomers of FIG. 7A.

FIG. 9A sets forth generalized synthetic schemes for synthesis of monomers from drugs or agents including amino groups.

FIG. 9B sets forth generalized synthetic schemes for synthesis of monomers from drugs or agents including carboxyl groups.

### DETAILED DESCRIPTION

It will be readily understood that the components of the embodiments, as generally described and illustrated in the figures herein, may be arranged and designed in a wide variety of different configurations in addition to the described representative embodiments. Thus, the following more detailed description of the representative embodiments, as illustrated in the figures, is not intended to limit the scope of the embodiments, as claimed, but is merely illustrative of representative embodiments.

Reference throughout this specification to “one embodiment” or “an embodiment” (or the like) means that a particular feature, structure, or characteristic described in connection with the embodiment is included in at least one embodiment. Thus, the appearance of the phrases “in one embodiment” or “in an embodiment” or the like in various places throughout this specification are not necessarily all referring to the same embodiment.

Furthermore, described features, structures, or characteristics may be combined in any suitable manner in one or more embodiments. In the following description, numerous specific details are provided to give a thorough understanding of embodiments. One skilled in the relevant art will recognize, however, that the various embodiments can be practiced without one or more of the specific details, or with other methods, components, materials, et cetera. In other instances, well known structures, materials, or operations are not shown or described in detail to avoid obfuscation.

As used herein and in the appended claims, the singular forms “a,” “an”, and “the” include plural references unless the context clearly dictates otherwise. Thus, for example, reference to “a hydrophilic compound” or “an immunotherapy agent” includes a plurality of such compounds or agents and equivalents thereof known to those skilled in the art, and so forth, and reference to “the hydrophilic compound” or “the immunotherapy agent” is a reference to one or more such compounds or agents and equivalents thereof known to those skilled in the art, and so forth. Recitation of ranges of values herein are merely intended to serve as a shorthand method of referring individually to each separate value falling within the range. Unless otherwise indicated herein, and each separate value, as well as intermediate ranges, are incorporated into the specification as if individually recited herein. All methods described herein can be performed in any suitable order unless otherwise indicated herein or otherwise clearly contraindicated by the text.

Targeted drug delivery via nanocarriers is an effective approach to improving the treatment of chemotherapeutic and other therapeutic agents. As used herein, the term chemotherapy agent, refers to a chemical substance used in vivo for the treatment and/or prevention of disease (for example, the treatment of cancer by cytostatic, cytotoxic and other drugs). However, very often, only limited efficacy can be achieved because of, for example, the development of multiple drug-resistance mechanisms. Combination of immune therapy with chemotherapy represents an attractive strategy to further improve the outcome of treatment as immune therapy kills tumor cells via mechanisms that are distinct from that of chemotherapy. Currently most of immunochemotherapy regimens involve the simple combination of different treatment protocols that are not only inconvenient but also of limited effectiveness. It is important to develop a strategy or strategies that is/are capable of simultaneous delivery of both immuno- and chemo-components to tumor tissues. As used herein, the term “immunotherapy agent” refers to a chemical substance which restores or stimulates an immune response for the treatment and/or prevention of disease. Immunotherapeutic agents hereof can be drugs or prodrugs. In a number embodiments, the immunotherapy agent is affective to restore or stimulate an immune response to treat or prevent cancer. In a number of embodiments, the immunotherapy agent operates synergistically with a chemotherapy agent (for example, with the co-delivered or another chemotherapy agent) and/or with radiotherapy. Further, immunotherapy agents hereof including polymerized forms of immunotherapy drugs or prodrugs

We have shown that IDO is significantly upregulated in tumor tissues following treatment with TAXOL. As described above, immunotherapy strategies, including those that are targeted at IDO represent an attractive approach for the treatment of cancer, particularly in combination with chemotherapy. Various IDO inhibitors have been reported, among which NLG919 is a highly IDO-selective inhibitor with an EC50 of 75 nM. However, most IDO inhibitors, including NLG919, are poorly water soluble and their in vivo applications require complicated protocols. Co-delivery of IDO inhibitor and chemotherapeutic agents to a tumor is a significant challenge because of their different physical and pharmacokinetic profiles.

A hydrophobic drug such as a hydrophobic immunotherapy agent, properly designed, can be converted to a drug carrier for other drugs via combination with a hydrophilic compound or domain (for example, via polyethylene glycol or PEG derivatization) while maintaining the pharmacological activity of the parent compound. In a number of representative embodiments hereof, we designed a PEG-NLG919 conjugate as a representative dual functional carrier to achieve effective codelivery of NLG919 and a chemotherapeutic drug to tumors. In a number of embodiments, we also introduced drug-interactive group or moiety such as a fluorenylmethyloxycarbonyl or Fmoc group into PEG-NLG919 conjugate. A drug-interactive group such as Fmoc functions as a “formulation chemophor” or a structural unit capable of interacting with many pharmaceutical agents. Drug carriers including drug-interactive groups are described in PCT International Patent Application Publication No. WO 2014/093631 and U.S. patent application Ser. No. 14/625,873, the disclosures of which are incorporated herein by reference. Drug-interactive groups suitable for user herein include, for example, a fluorenylmethyloxycarbonyl group, a carbobenzyloxy group, an isobutoxycarbamate group, a naphthylacetyl group, a carbazole group, a quinolone group, an isoquinolone group, or a group which is a residue of a molecule selected from the group of the compound, a portion of the compound, (9H-fluoren-9-yl)methanamine, (9H-fluoren-9-yl)methanol, 9H-fluoren-9-amine, naphthalene, 1,1′-bi-2-naphthol (BINOL), camptothecin, a camptothecin analog, pemetrexed, docetaxel, paclitaxel, epirubicin, doxorubicin, vinblastine, vindesine, etoposide, hydroxycamptothecin, irinotecan, mitoxantrone, tamoxifen, tretinoin, Vitamin A, Vitamin E, Vitamin K, Vitamin D, curcumin, imatinib, gefitinib, erlotinib, sorafenib, and bortezomib, or a derivative thereof. Fluorenylmethyloxycarbonyl or Fmoc groups and derivatives thereof are particularly suited for use as compound-interactive or drug-interactive groups.

As described above, in a number of embodiments, an interfacial region of an amphiphilic agent/molecule including at least one hydrophobic immunotherapy agent (drug/prodrug) domain and at least one hydrophilic domain is modified (for example, enlarged and/or expanded) by inserting an drug- or compound-interactive segment. Such interactive sections may, for example, include interactive groups such as amino acid or a peptide segments. Additionally, pendant groups on the amino acid or other residues may be incorporated that exhibit drug-interactive potential. Pendant and/or other groups of the compound/drug-interactive segments, regions or domains hereof may, for example, be capable of π-π hydrophobic/aromatic ring stacking or hydrogen-bonding interactions to enhance the carrier-drug interaction as a way to stabilize drug formulation.

The compound/drug-interactive segment, region or domain may, for example, be experimentally determined through, for example, solubility tests of individual motifs. The mode of detection may, for example, be visual (for example, under a microscope) for the suppression/disappearance of crystal formation, by optical density (OD) reading, by high pressure liquid chromatography (HPLC) or any other suitable measurement method for the soluble fraction of a poorly water soluble free drug that is facilitated to form nanostructure a solution in aqueous solutions. The compound or a portion of the compound with which the interactive segment, region or domain is to interact can also be used in the interactive segments, regions or domains. For example, reactive groups on the compound or a portion thereof (either native to the compound/portion or created thereon by modification) can be used to bond a residue of the compound/portion within the carrier agent. Motifs immobilized on solid phase support may, for example, also be useful for the identification process by, for example, binding or absorbing a particular agent to be tested compared to the unmodified solid phase support.

The motifs may, for example, additionally or alternatively be predicted theoretically based on the known structural features of a particular agent, such as charge properties, aromatic ring structures, hydrogen bonding potential, etc.

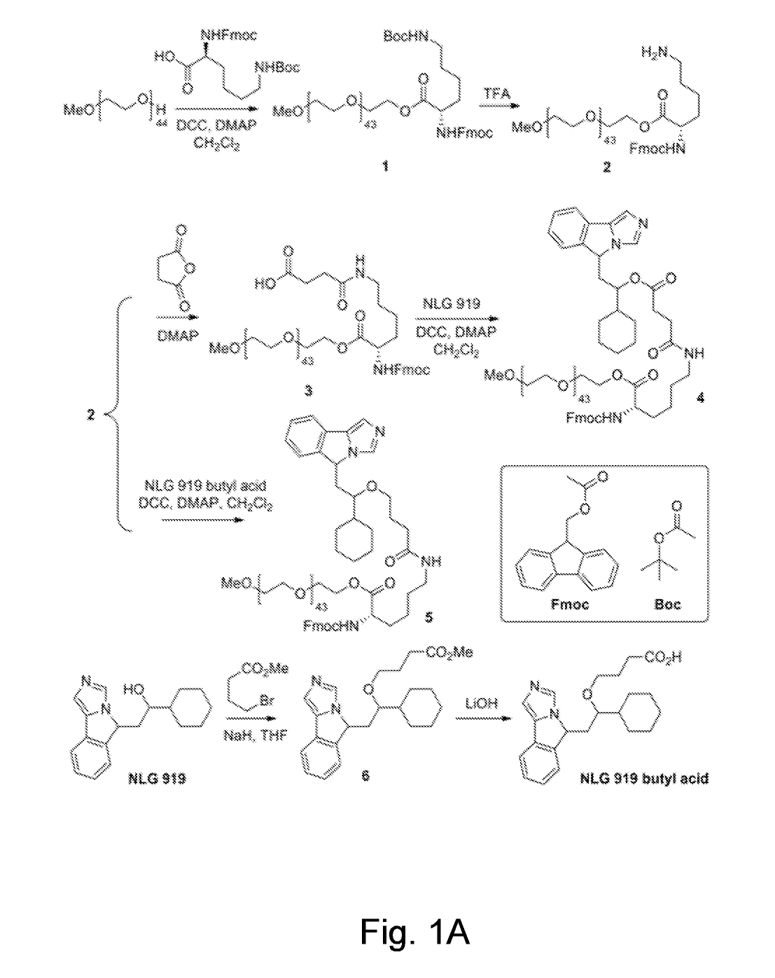

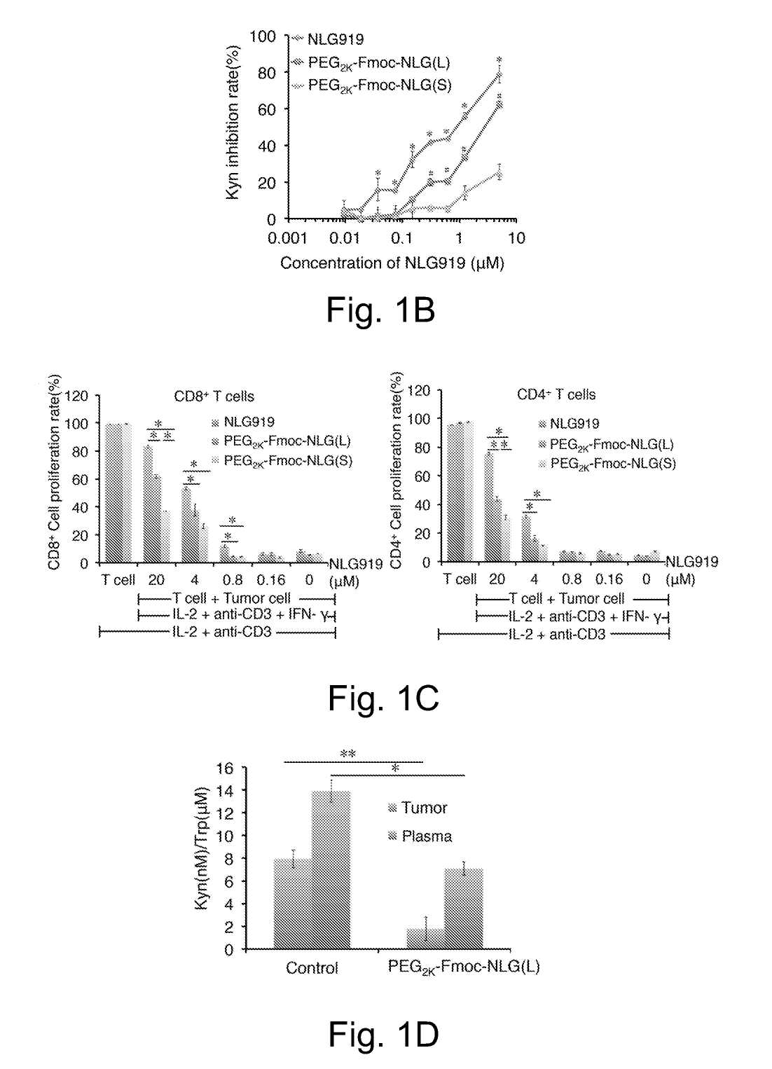

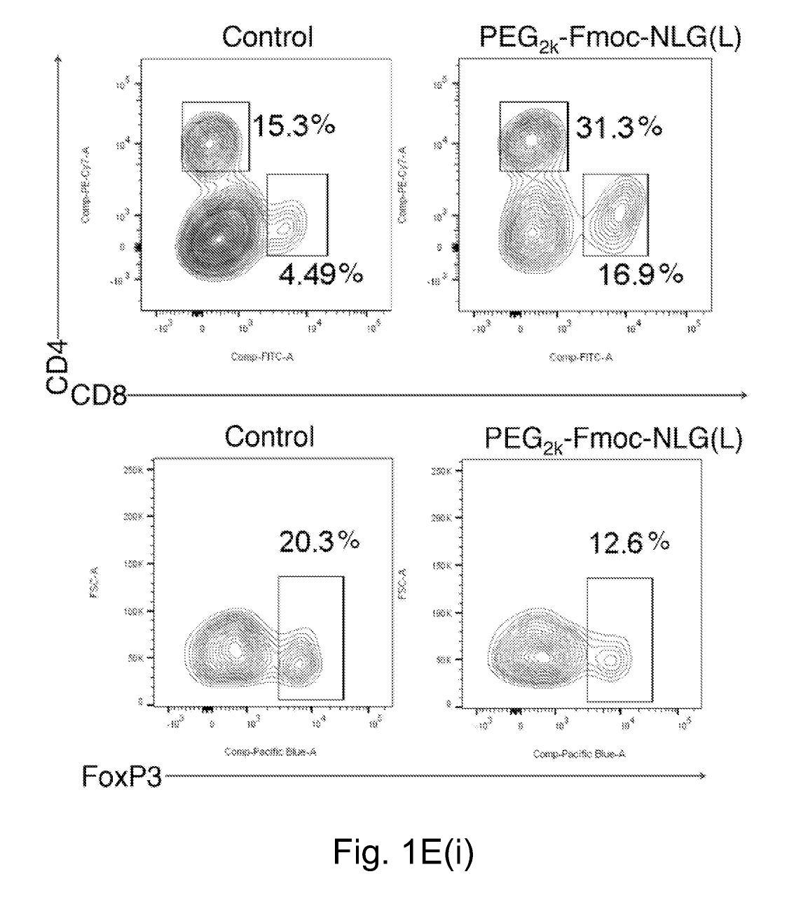

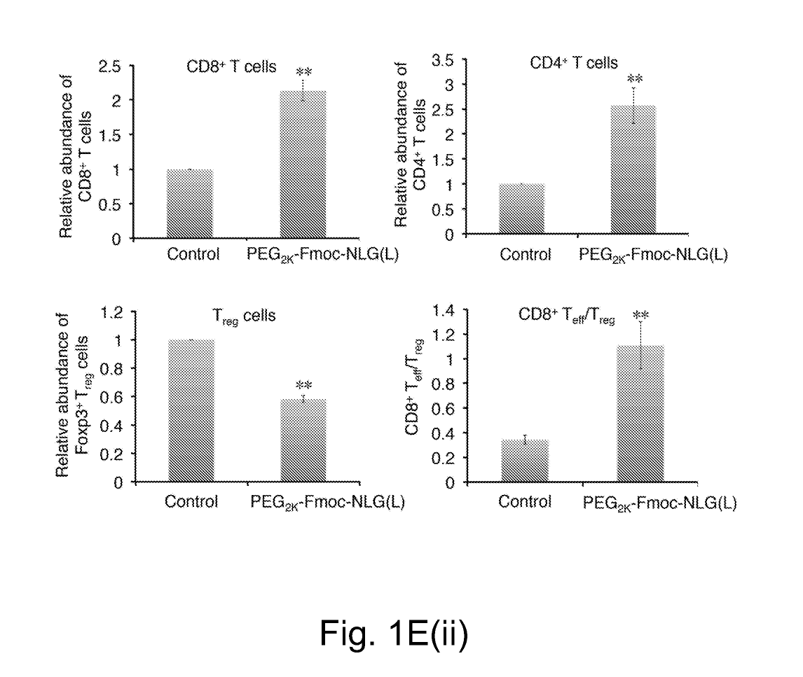

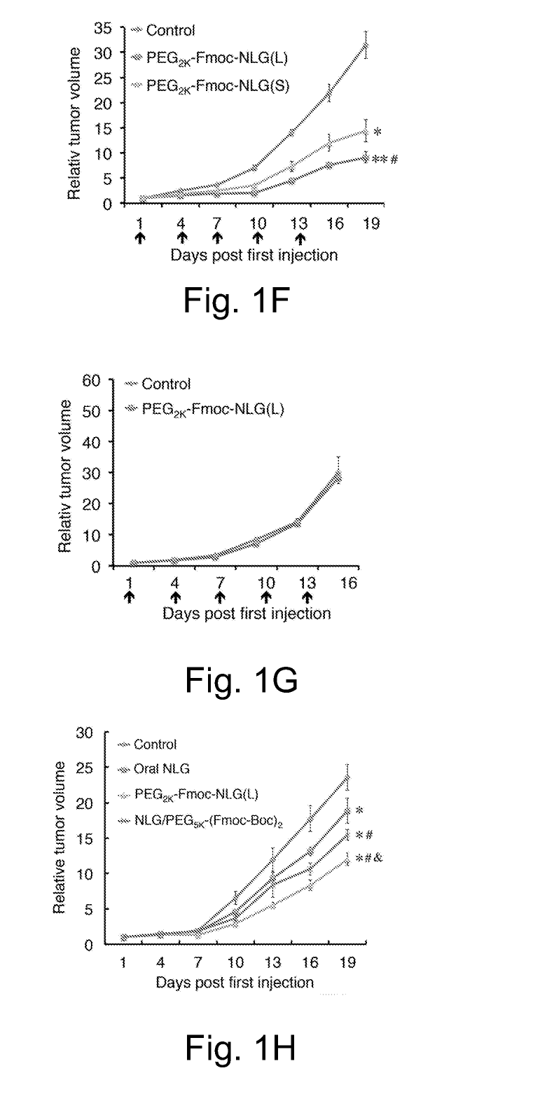

PEG₂ₖ-Fmoc-NLG is an amphiphilic molecule that self-assembles into micelles in aqueous solutions into which hydrophobic drugs may be loaded. Incorporation of an Fmoc motif (or other drug-interactive motif) as described above into a micellar or other system may not only improve the drug loading capacity and formulation stability but also broaden its utility in formulating various therapeutic agents of diverse structures. FIG. 1A shows a representative embodiment of a synthesis scheme of two representative PEG2K-Fmoc-NLG conjugates, one with a relatively labile ester linkage (PEG2K-Fmoc-NLG(L)) and the other one with a relatively stable amide linkage (PEG2K-Fmoc-NLG(S)). The chemical structures of the two conjugates were confirmed by NMR and mass spectrometry (MS).

The inhibitory activity of PEG₂ₖ-Fmoc-NLG(L) and PEG₂ₖ-Fmoc-NLG(S) on IDO was evaluated by examining their potency in inhibiting the conversion of Trp to kynurenine (Kyn) in HeLa cells. HeLa cells were treated with IFN-γ to induce IDO expression and the amounts of Trp and Kyn in culture medium were determined by a colorimetric assay. As shown in FIG. 1B, free NLG919 inhibited the IDO activity in a concentration-dependent manner with an EC50 of 0.95 μM. PEG₂ₖ-Fmoc-NLG(L) was less active (EC50 of 3.4 μM) in inhibiting IDO compared to free NLG919 while PEG₂ₖ-Fmoc-NLG(S) was least active (EC50>10 μM). Similar results were obtained when the Trp and Kyn concentrations were measured by LC/MS. We then examined if inhibition of IDO by PEG₂ₖ-Fmoc-NLG(L) led to enhanced T cell proliferation in an in vitro lymphocyte and Panc02 (a murine pancreatic cancer cell line) coculture experiment. As shown in FIG. 1C, coculture of IDO⁺ tumor cells with splenocytes isolated from BALB/c mice led to significant inhibition of T cell proliferation. This inhibition was significantly attenuated when the mixed cells were treated with NLG919. PEG₂ₖ-Fmoc-NLG(L) was also active in reversing the inhibitory effect of tumor cells although slightly less potent than NLG919. PEG₂ₖ-Fmoc-NLG(S) is less active compared to PEG₂ₖ-Fmoc-NLG(L) (FIG. 1C).

The formulations hereof may, for example, form a complex such as, for example, a micelle, an emulsion, a cream, a liposome, a spherulite, a solid-lipid nanoparticle, a hydrogel or a cubic phase lipogel. Lipidic based formulations, such as liposomes, emulsions and micelles, are attractive drug delivery systems for in vivo applications because of their excellent safety profiles.

The in vivo biological activity of PEG₂ₖ-Fmoc-NLG(L) was evaluated in an aggressive murine breast cancer model, 4T1.2. PEG₂ₖ-Fmoc-NLG(L) self-assembled to form nano-sized micelles (˜90 nm) in aqueous solutions, which enable effective and selective delivery to tumors via enhanced permeation and retention (EPR) effect. As shown in FIG. 1C, the ratios of Kyn (nM)/Trp (μM) in both blood and tumors were significantly reduced following the treatment of PEG₂ₖ-Fmoc-NLG(L) while a more dramatic reduction was observed in the tumor tissues, consistent with the intended specific targeting of IDO inhibitors to the tumor tissues. FIG. 1E shows multi-color flow cytometric analysis of tumor-infiltrating lymphocytes in 4T1.2 tumor-bearing mice with or without treatment of PEG₂ₖ-Fmoc-NLG(L). It is clear that more CD4⁺ and CD8⁺ T cells were found in the tumors that received the treatment of PEG₂ₖ-Fmoc-NLG(L). In addition, the number of regulatory T cells (Tregs) was significantly reduced in the tumors treated with PEG₂ₖ-Fmoc-NLG(L).

FIG. 1F shows the in vivo antitumor activity of PEG₂ₖ-Fmoc-NLG(L) and PEG₂ₖ-Fmoc-NLG(S) in 4T1.2 tumor model. Significant antitumor responses were observed for both prodrugs. It is also apparent that PEG₂ₖ-Fmoc-NLG(L) was more effective than PEG₂ₖ-Fmoc-NLG(S) in inhibiting the tumor growth. We also showed that PEG₂ₖ-Fmoc-NLG(L) was essentially not active in inhibiting the growth of 4T1.2 tumor in the immunocompromised nude mice that lack T and B cells (FIG. 1G), indicating, without limitation to any particular mechanism, that the antitumor response was mediated via an enhanced T cell immune response. The above data demonstrated that PEG-derivatized NLG919 prodrug well retained the pharmacological activity of NLG919 and that the cleavability of NLG919 from the conjugate affected its activity. We have further shown that i.v. PEG₂ₖ-Fmoc-NLG(L) was more effective than NLG919 delivered orally (FIG. 1H). In addition, i.v. PEG₂ₖ-Fmoc-NLG(L) was more active than an i.v. formulation of NLG919 that was loaded into PEG₅ₖ-(Fmoc-Boc)₂ micelles (FIG. 1H).

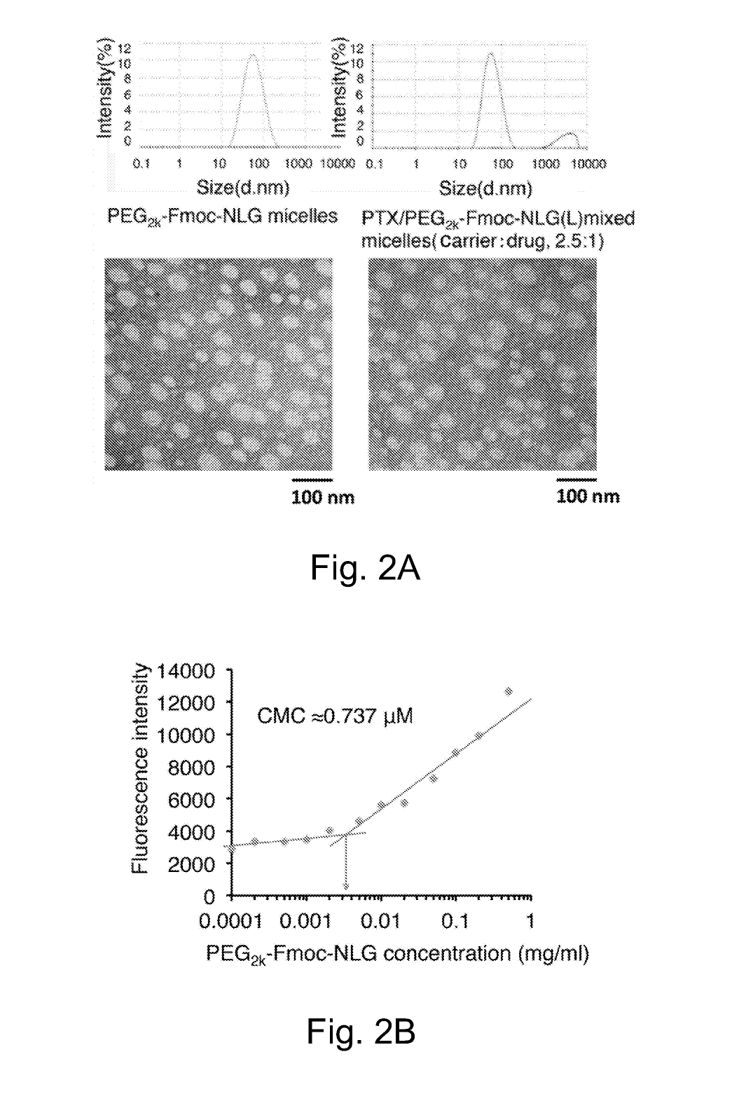

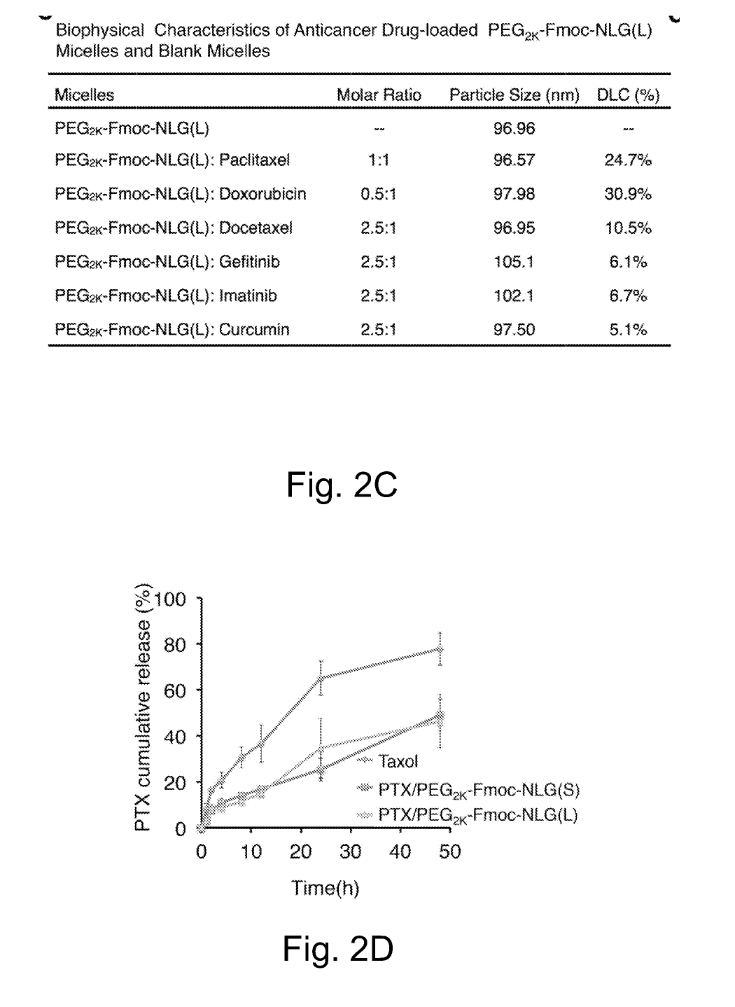

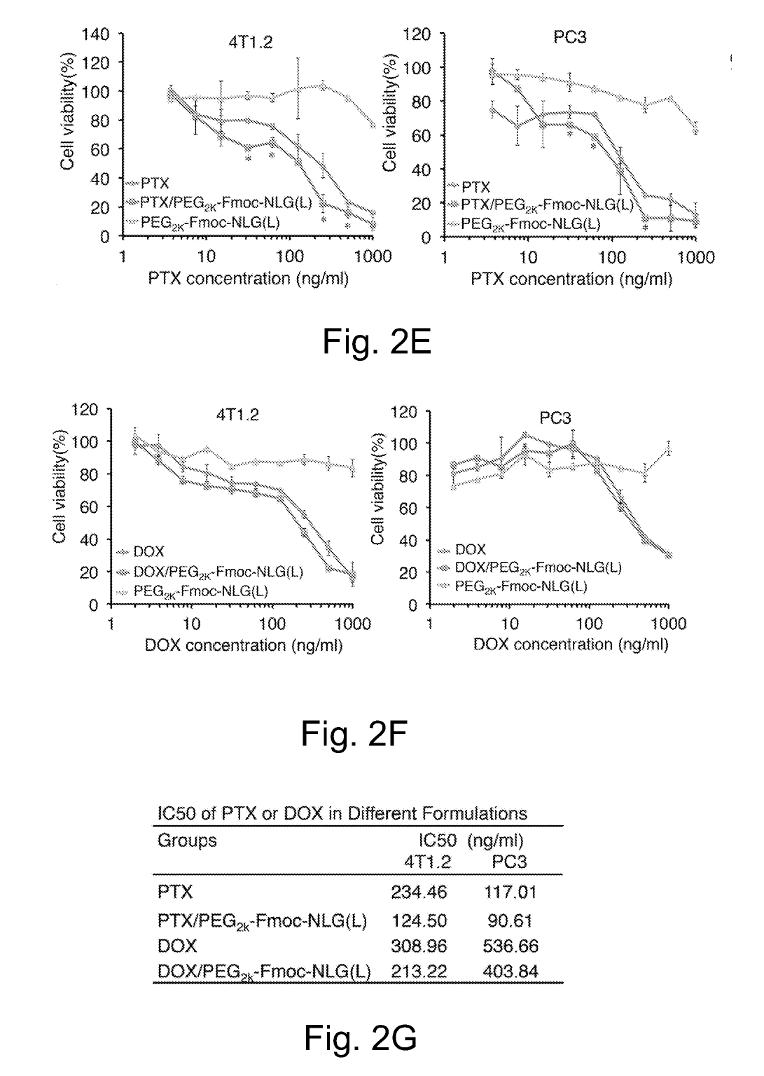

As described above, PEG₂ₖ-Fmoc-NLG(L) readily formed small-sized (˜90 nm) micelles in aqueous solutions as confirmed by DLS and TEM imaging (FIG. 2A). Loading of paclitaxel (PTX) into PEG₂ₖ-Fmoc-NLG(L) micelles resulted in minimal changes in the sizes of the particles and their morphology (FIG. 2A). Similar results were obtained for PEG₂ₖ-Fmoc-NLG(S) micelles (data not shown). FIG. 2B shows that the critical micelle concentration (CMC) of PEG₂ₖ-Fmoc-NLG(L) was 0.737 μM. The relatively low CMC may render the micelles stable upon dilution in the blood, which is important for systemic delivery to tumors. FIG. 2C shows the drug loading capacity (DLC) of PEG₂ₖ-Fmoc-NLG(L) for several commonly used chemotherapeutic agents including PTX, docetaxel, doxorubicin (DOX), gefitinib, imatinib, and curcumin. Without limitation to any mechanism, the effectiveness of PEG₂ₖ-Fmoc-NLG(L) in formulating various anticancer agents of diverse structures may, for example, be attributed to the strong carrier/drug interactions including hydrophobic/hydrophobic interaction, π-π stacking and hydrogen bonding.

FIG. 2D shows the kinetics of PTX release from PTX/PEG₂ₖ-Fmoc-NLG in comparison with Taxol. Taxol showed a relatively fast release of PTX with greater than 60% of PTX being released within the 1^{st} 24 h. Close to 80% of PTX was released from Taxol after 48 h. In contrast, the kinetics of PTX release was significantly slower for either PTX/PEG₂ₖ-Fmoc-NLG(L) or PTX/PEG₂ₖ-Fmoc-NLG(S) formulation. Only 20-30% of PTX was released within the 1^{st} 24 h and more than 50% of the PTX remained associated with the micelles after 48 h. Nonetheless, release of PTX from either PTX/PEG₂ₖ-Fmoc-NLG(L) or PTX/PEG₂ₖ-Fmoc-NLG(S) was significantly faster than the cleavage and release of NLG from either carrier; free NLG was essentially undetectable during the entire release study.

FIG. 2E shows the cytotoxicity of PTX-loaded PEG₂ₖ-Fmoc-NLG(L) in 4T1.2 cells. PEG₂ₖ-Fmoc-NLG(L) alone was not effective in inhibiting the tumor cell growth at the test concentrations. Free PTX inhibited the tumor cell growth in a concentration-dependent manner. PTX-loaded PEG₂ₖ-Fmoc-NLG(L) micelles were more effective (P<0.05) than free PTX at several concentrations tested (FIG. 2E). Similar results were found in the PC3 human prostate cancer cell line (FIG. 2E). We also observed enhanced cytotoxicity (P=0.053) for DOX following incorporation into PEG₂ₖ-Fmoc-NLG(L) micelles (FIG. 2F). The IC50s of free drugs (PTX or DOX) and drug-loaded micelles are shown in FIG. 2G.

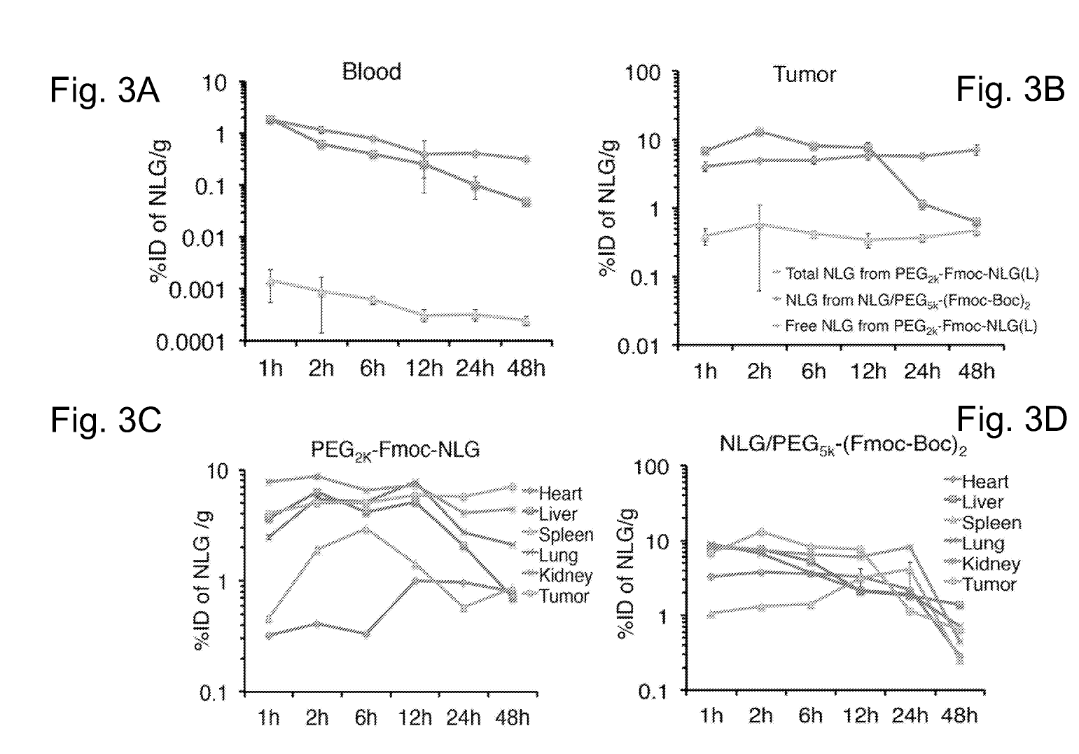

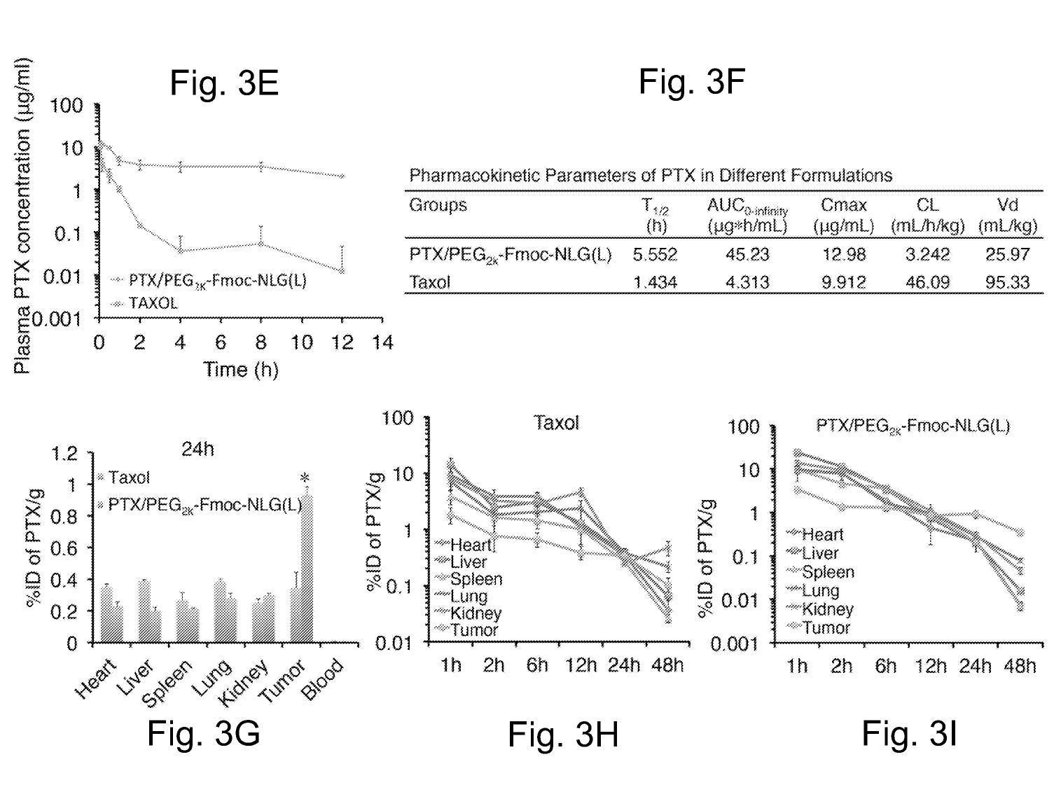

FIG. 3A shows the kinetics of PEG-Fmoc-NLG in the blood in comparison to NLG loaded into PEG₅ₖ-(Fmoc-Boc)₂ micelles. The concentrations of total NLG (intact PEG₂ₖ-Fmoc-NLG plus released free NLG) in the blood were significantly higher than the blood concentrations of NLG delivered by PEG₅ₖ-(Fmoc-Boc)₂ micelles at most time points examined. It is also apparent that very little free NLG was detected in the blood in the group treated with PEG₂ₖ-Fmoc-NLG, suggesting the excellent stability of the conjugate in the blood.

FIG. 3B shows the amounts of total NLG in the tumors at different time points following i.v. administration of either PEG₂ₖ-Fmoc-NLG or NLG-loaded PEG₅ₖ-(Fmoc-Boc)₂ micelles. The NLG concentrations in the tumors in NLG/PEG₅ₖ-(Fmoc-Boc)₂ group reached the peak levels at 2 h and then quickly declined over time. In contrast, high concentrations of NLG (largely intact conjugate) were found in the tumors over the entire 48 h in the mice treated with PEG₂ₖ-Fmoc-NLG. It is also apparent that a relatively constant concentration of free NLG was detected in the tumors in this group, albeit at a low level, suggesting that NLG was slowly but continuously released from the conjugate over a prolonged period of time. FIGS. 3C and 3D show the total amounts of NLG in tumors and other major organs/tissues at various times following i.v. administration of either PEG₂ₖ-Fmoc-NLG or NLG/PEG₅ₖ-(Fmoc-Boc)₂ mixed micelles.

FIG. 3E shows the blood PTX kinetics in BALB/c mice as a function of time following i.v. bolus administration of PTX-loaded PEG₂ₖ-Fmoc-NLG(L) and Taxol. It is apparent that PTX/PEG₂ₖ-Fmoc-NLG(L) remained in the circulation for a significantly longer time compared to Taxol. The pharmacokinetic parameters are outlined in FIG. 3F. Incorporation of PTX into PEG₂ₖ-Fmoc-NLG(L) micelles resulted in significantly greater t_{1/2}, AUC, and Cₘₐₓ over Taxol. Meanwhile, Vd and CL for PTX/PEG₂ₖ-Fmoc-NLG(L) were significantly lower than those for Taxol.

FIG. 3G shows the biodistribution of PTX in 4T1.2 tumor-bearing mice 24 h following i.v. administration of PTX-loaded PEG₂ₖ-Fmoc-NLG(L) micelles or Taxol. Significantly greater amounts of PTX were found in tumor tissues for PTX-loaded PEG₂ₖ-Fmoc-NLG(L) micelles in comparison with Taxol. In contrast, PTX-loaded PEG₂ₖ-Fmoc-NLG(L) micelles showed significantly reduced accumulation than Taxol in liver, spleen and other organs/tissues. These data strongly suggest that PTX-loaded PEG₂ₖ-Fmoc-NLG(L) micelles are stable in the blood and are highly effective in selective delivery to the tumor tissues. FIGS. 3H and 3I show the amounts of PTX in tumors and other major organs/tissues at various times following i.v. administration of either PTX-loaded PEG₂ₖ-Fmoc-NLG(L) micelles or Taxol.

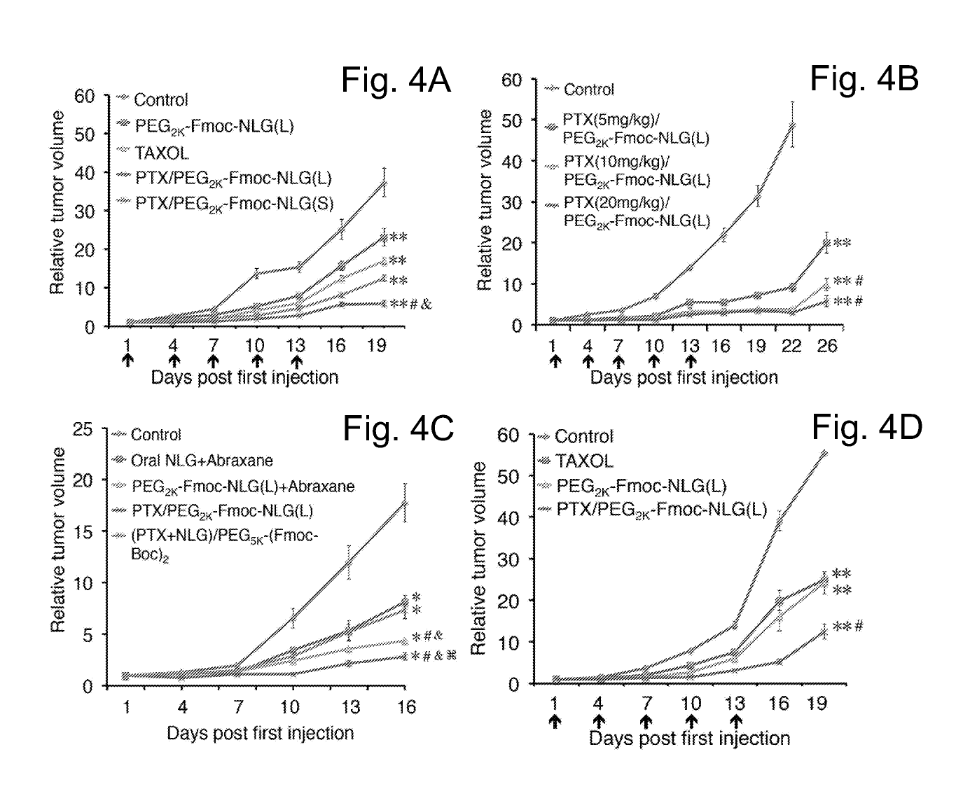

FIG. 4A shows the in vivo antitumor activity of PEG₂ₖ-Fmoc-NLG(L), Taxol, PTX/PEG₂ₖ-Fmoc-NLG(S), and PTX/PEG₂ₖ-Fmoc-NLG(L) at a PTX dosage of 10 mg/kg. Taxol showed a modest effect in inhibiting the growth of 4T1.2 tumor, which was comparable to that of PEG₂ₖ-Fmoc-NLG(L) alone. However, both PTX/PEG₂ₖ-Fmoc-NLG(S) and PTX/PEG₂ₖ-Fmoc-NLG(L) were more effective than Taxol or PEG₂ₖ-Fmoc-NLG(L) in inhibiting the tumor growth. PTX/PEG₂ₖ-Fmoc-NLG(L) was more effective than PTX/PEG₂ₖ-Fmoc-NLG(S), indicating a potential role of released NLG919 in the overall antitumor activity of PTX/PEG₂ₖ-Fmoc-NLG(L). The antitumor activity of the three PTX formulations follows the order of PTX/PEG₂ₖ-Fmoc-NLG(L) >PTX/PEG₂ₖ-Fmoc-NLG(S) >Taxol≈PEG₂ₖ-Fmoc-NLG(L).

FIG. 4B shows the antitumor activity of PTX/PEG₂ₖ-Fmoc-NLG(L) at various doses of PTX. Tumor growth was well controlled at all dose groups at early time points. After the last treatment at day 13, the tumor growth was almost stalled until day 22 for the groups of 10 and 20 mg PTX/kg. After that, there was a rebound in tumor growth, particularly in the low dose group.

FIG. 4C shows that PTX/PEG₂ₖ-Fmoc-NLG(L) was also more effective than a combination therapy that involves oral delivery of NLG together with i.v. administration of Abraxane. In addition, PTX/PEG₂ₖ-Fmoc-NLG(L) was more active than a combination of i.v. Abraxane with i.v. PEG₂ₖ-Fmoc-NLG(L). Furthermore, PTX/PEG₂ₖ-Fmoc-NLG(L) was more active than an i.v. formulation of PEG₅ₖ-(Fmoc-Boc)₂ that was co-loaded with PTX and NLG. Improved antitumor activity of PTX/PEG₂ₖ-Fmoc-NLG(L) was also demonstrated in an aggressive B16 murine melanoma model (FIG. 4D).

All of the treatments were well tolerated by the mice and there were no abnormal physical signs in all treated mice. In addition, there were no obvious differences among all of the groups in body weights in all of the different therapy studies.

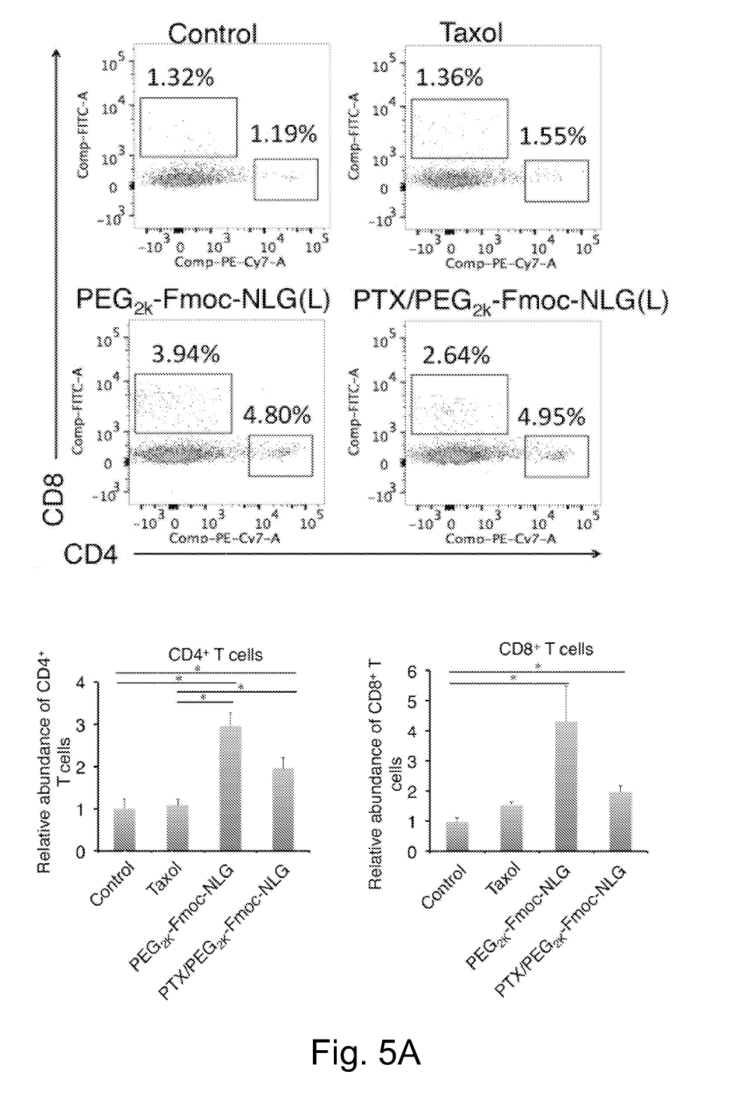

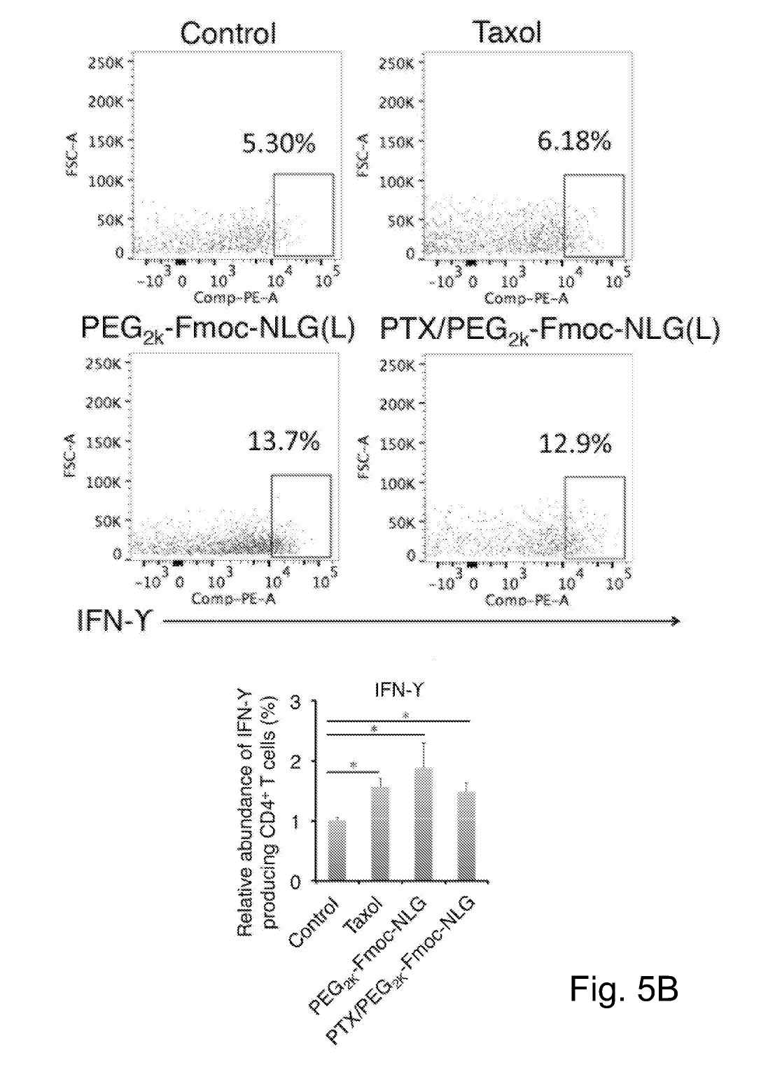

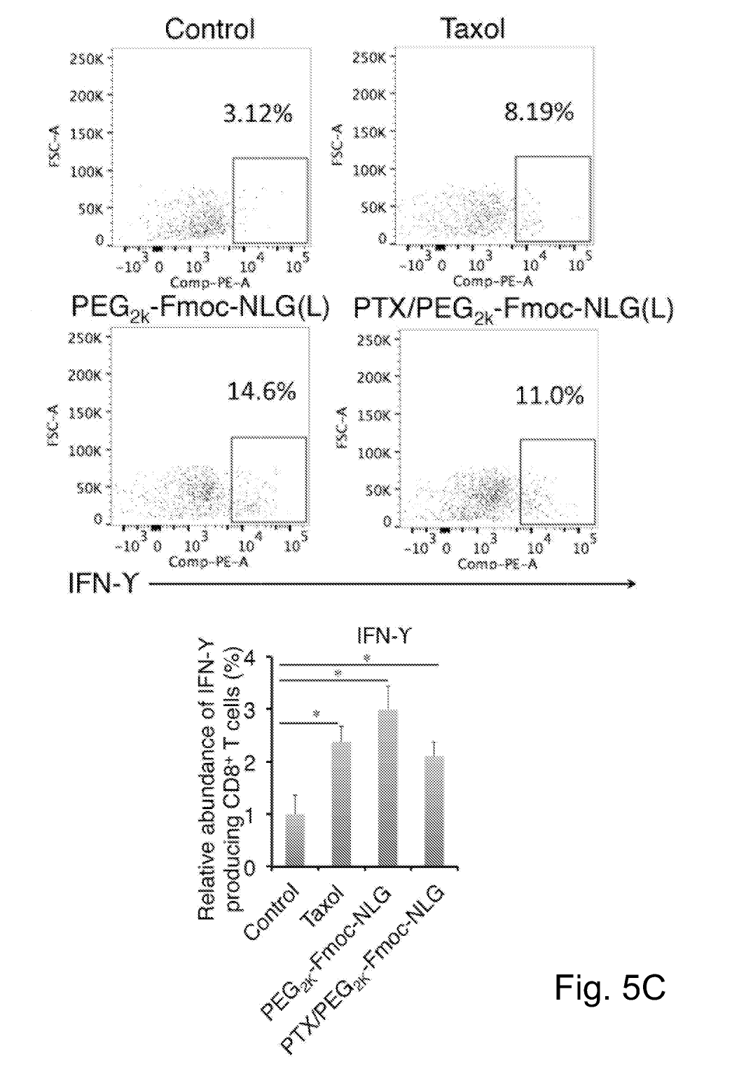

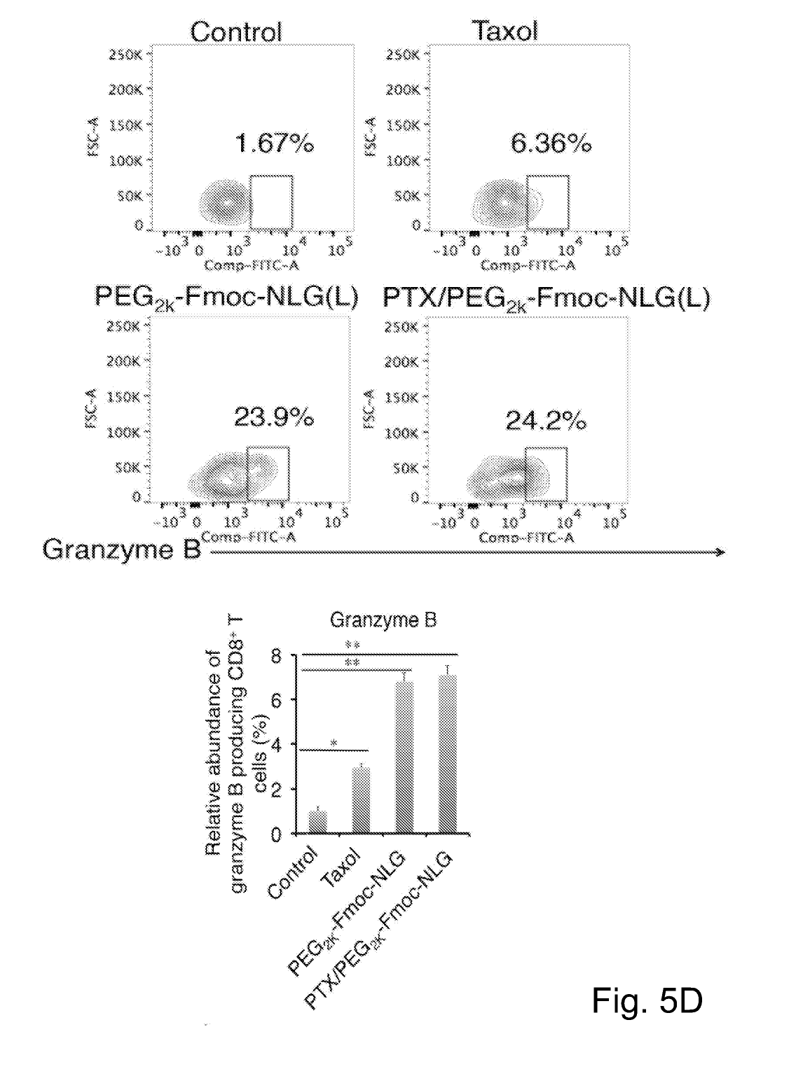

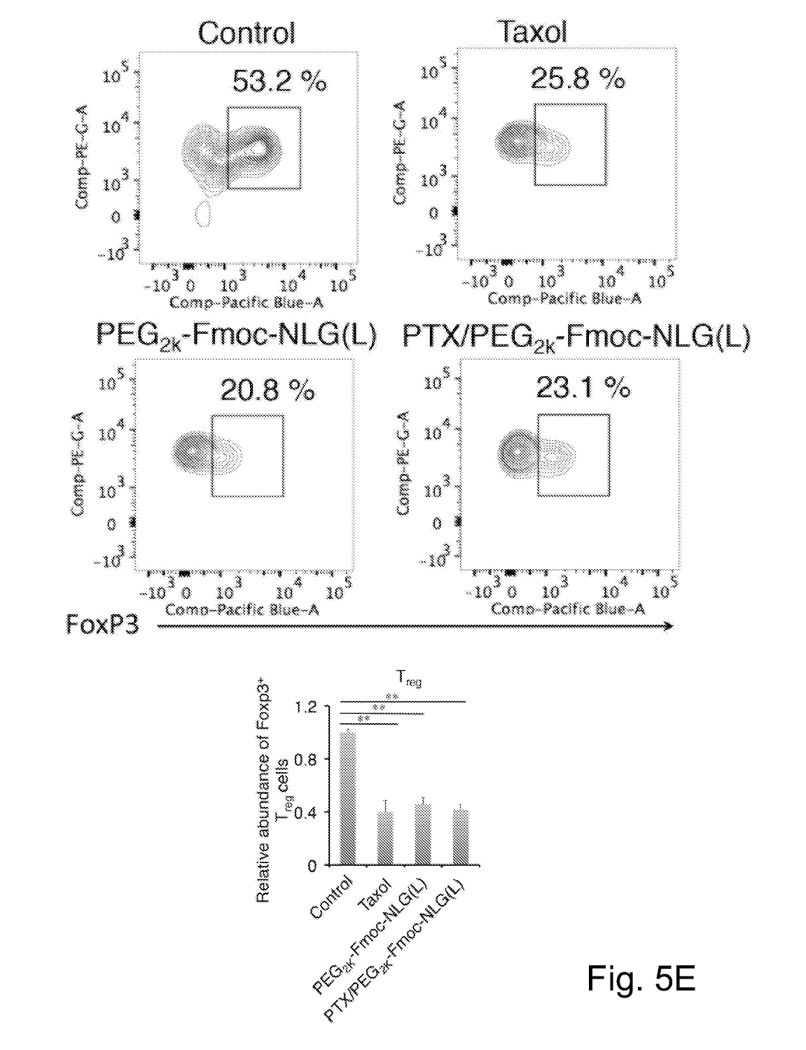

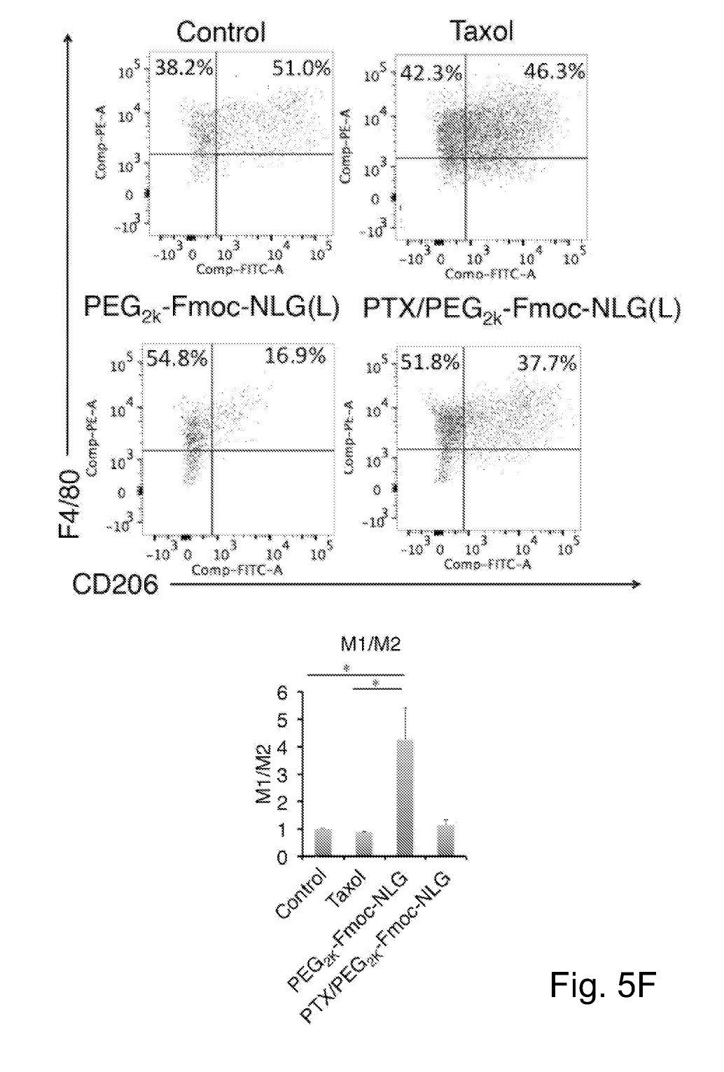

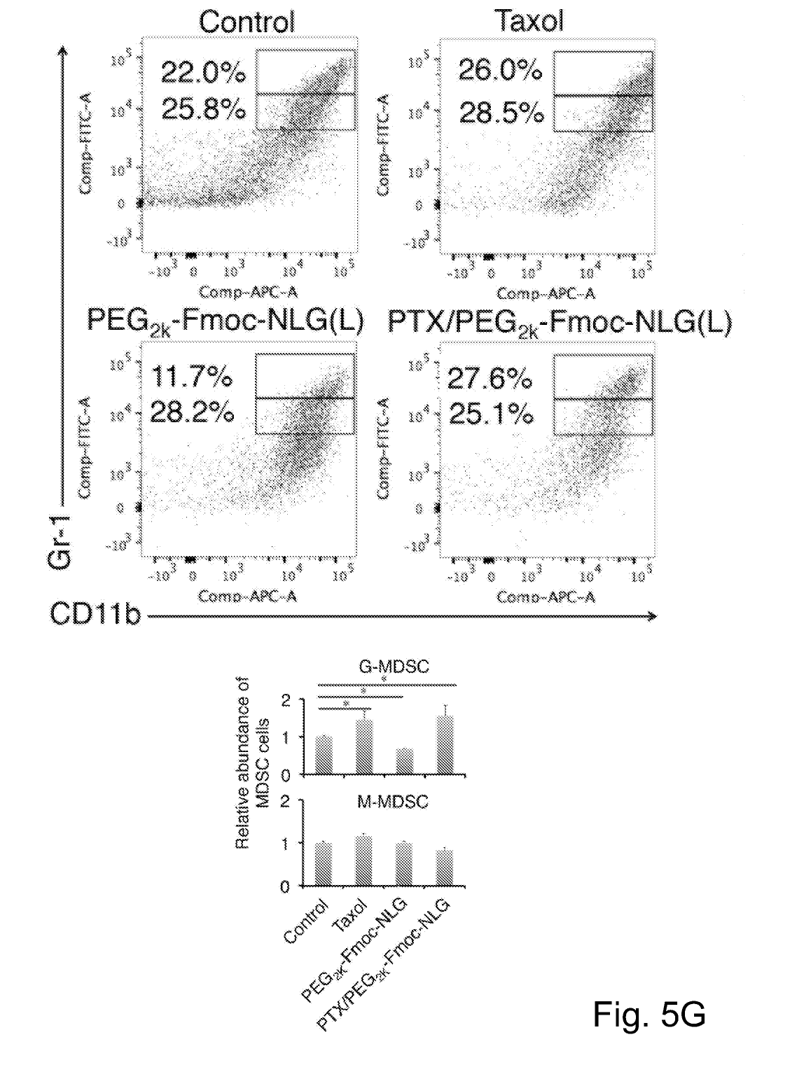

To delineate a role of immune response in PTX/PEG₂ₖ-Fmoc-NLG(L)-mediated antitumor activity, the immune cell populations in the tumor tissues with various treatments were analyzed by flow cytometry one day following 5 times of treatments. FIG. 5A shows infiltration of more CD4⁺ T cells in the tumors treated with PTX/PEG₂ₖ-Fmoc-NLG(L) compared to control or Taxol groups (P<0.05). There were also more CD8⁺ T cells in the tumors treated with PTX/PEG₂ₖ-Fmoc-NLG(L) compared to control group. It was also noted that the numbers of both CD4⁺ and CD8⁺ T cells in Taxol-treated tumors were lower than those in the tumors treated with carrier alone (FIG. 5A). Delivery of PTX via PEG₂ₖ-Fmoc-NLG(L) was associated with a similar reduction in the numbers of CD4⁺ and CD8⁺ T cells (FIG. 5A).

FIGS. 5B and 5C show that the numbers of IFN-γ-positive CD4⁺ or CD8⁺ T cells were significantly increased in the tumors treated with Taxol, PEG₂ₖ-Fmoc-NLG(L) or PTX/PEG₂ₖ-Fmoc-NLG(L). The magnitude of increase was similar among all of the treatment groups.

The numbers of granzyme B-positive CD8⁺ T cells were also significantly increased in all of the treatment groups (FIG. 5D). However, there were significantly more granzyme B-positive CD8⁺ T cells in the tumors treated with PEG₂ₖ-Fmoc-NLG(L) or PTX/PEG₂ₖ-Fmoc-NLG(L) compared to Taxol-treated tumors (FIG. 5D). There were no differences between PEG₂ₖ-Fmoc-NLG(L) and PTX/PEG₂ₖ-Fmoc-NLG(L) groups in the numbers of granzyme B-positive CD8⁺ T cells (FIG. 5D). Treg cells were significantly decreased in all treatment groups compared to control group (P<0.01) and there were no significant differences among these treatment groups (P>0.05) (FIG. 5E).

FIG. 5F shows that the M1/M2 ratios of tumor-associated macrophages were significantly increased in the tumors treated with PEG₂ₖ-Fmoc-NLG(L). The M1/M2 ratios in the tumors treated with Taxol or PTX/PEG₂ₖ-Fmoc-NLG(L) were similar to those in the control group.

FIG. 5G shows that the numbers of granulocytic myeloid derived suppressor cells (G-MDSC) were significantly decreased in the tumors treated with PEG₂ₖ-Fmoc-NLG(L) alone. This is consistent with the previous reports that inhibition of IDO leads to decreased MDSC in the tumors. Surprisingly, G-MDSC were significantly increased in the tumors treated with either PTX/PEG₂ₖ-Fmoc-NLG(L) or Taxol. There were no significant differences among all of the groups in the numbers of monocytic MDSC (M-MDSC) in the tumors (FIG. 5G).

The histology of tumors at the time of flow cytometry analysis showed that tumors from the mice treated with PTX/PEG₂ₖ-Fmoc-NLG(L) exhibited significant necrosis/apoptosis of tumor cells. Tumors treated with Taxol or PEG₂ₖ-Fmoc-NLG also showed moderate tumor cell damage. Overall, the above data suggest that the microenvironment in the tumors treated with PTX/PEG₂ₖ-Fmoc-NLG(L) was more immune-active than that in Taxol-treated tumors. These results are consistent with the data that the in vivo IDO activity was more effectively inhibited in mice treated with PTX/PEG₂ₖ-Fmoc-NLG(L) compared to Taxol-treated mice.

The above data demonstrated that the representative PEG-derivatized NLG919 prodrug well retained the pharmacological activity of NLG919 and that the cleavability of NLG919 from the conjugate affected its activity. In addition to solving the issue of poor water solubility, PEG-NLG919 and other conjugates hereof may also serve as a depot system to achieve sustained release over a prolonged period of time. The linkage may, for example, be modulated to control the timing of release. In that regard, some linkages are more readily cleaved than others. Moreover, neighboring steric hindrance may, for example, be adjusted to control cleaving/release.

Different from most drug carriers that are “inert”, PEG-Fmoc-NLG and other carriers or carrier agents hereof are prodrugs that exhibits immunostimulatory activity. Despite its reduced EC50 compared to free NLG with respect to the potency in inhibiting IDO in cultured cells, PEG-Fmoc-NLG was significantly more effective than NLG that was formulated in a similar “inert” nanocarrier without a NLG motif (PEG₅ₖ-(Fmoc-Boc)₂ ) (FIG. 1H). In addition, i.v. PEG-Fmoc-NLG was more active than NLG delivered orally (FIG. 1H). This observation may, for example, be the result of the effective delivery of PEG-Fmoc-NLG to the tumors (FIGS. 3B, 3C and 3D). The slow release of NLG from PEG-Fmoc-NLG in tumor tissues (FIG. 3B) may also play a role.

A major advantage of the systems, methods and compositions hereof is simultaneous delivery to the tumors of two agents of different mechanisms of action. In addition, the systems hereof may, for example, provide a programmable release of various drug components via both chemical conjugation and physical encapsulation. PTX and NLG showed different temporal release kinetics upon codelivery to tumors. PTX has a much faster rate of release compared to that of NLG (FIG. 2D and 3B). PEG-Fmoc-NLG also has a longer retention time in the tumors (FIG. 3B), may, for example, be a result of its macromolecule nature. Delivery of PTX via PEG-Fmoc-NLG was more effective in inhibiting the tumor growth than codelivery of PTX and NLG via a similar “inert” nanocarrier without a NLG motif (FIG. 4C). In addition, PTX/PEG₂ₖ-Fmoc-NLG(L) was more effective than oral delivery of NLG together with i.v. administration of Abraxane (FIG. 4C). Without limitation to any particular mechanism, the relatively rapid release of PTX may, for example, lead to the first round of antitumor response that is further potentiated by the immune response that follows. The immune response may, for example, result from enhanced antigen presentation following PTX-mediated killing of tumor cells and/or direct effect of PTX on immune cells. Meanwhile, the slow release of active NLG919 from the prodrug may help in sustaining or enhancing the magnitude of immune responses by reversing IDO-mediated immune suppression. As a result, the combined therapy has produced a substantial inhibition of tumor growth. In fact, PTX/PEG₂ₖ-Fmoc-NLG(L) outperformed most reported PTX formulations including PTX formulated in our non-immunostimulatory dual functional carriers. It is possible that the carrier-mediated antitumor activity may be further improved via incorporation of a tumor microenvironment-responsive linkage to facilitate the NLG release. Another advantage of the systems, methods and compositions hereof lies in their simplicity with respect to both the synthesis of dual function carrier and the combination therapy protocol, which may, for example, facilitate a rapid translation into clinic. In addition, the nanocarriers hereof are versatile in formulating various anticancer agents of diverse structures (FIG. 2C).

Immunological analysis indicates a likely role of enhanced immune response in the overall antitumor activity of PTX/PEG₂ₖ-Fmoc-NLG(L). There were significantly more functional CD4⁺ and CD8⁺ cells in the tumors treated with PTX/PEG₂ₖ-Fmoc-NLG(L) compared to Taxol-treated tumors. However, we observed increased numbers of MDSC in both PTX/PEG₂ₖ-Fmoc-NLG(L) and Taxol groups, while the number of MDSC was significantly decreased in the group treated with carrier alone. PTX has been shown to be capable of reducing the number of both Treg and MDSC. We noticed significantly reduced numbers of Treg in both PTX/PEG₂ₖ-Fmoc-NLG(L)- and Taxol-treated groups, but observed the opposite effect on MDSC. This might, for example, be the result of the differences in the dose of PTX and the treatment regimen.

In a number of embodiments, compositions and methods hereof provide simple and effective immunochemotherapy approaches that are based on immunochemotherapy-mediated (for example, PEG-NLG919-mediated) codelivery of a chemotherapy agent such as PTX. The present approach ensures effective codelivery of the chemotherapy agent (for example, PTX) and the immunotherapy agent (for example, PEG-NLG prodrug) to the tumor in addition to solving the problem of in vivo application of both the chemotherapy agents and immunotherapy agents (for example, PTX and NLG919) arising from poor water solubility.

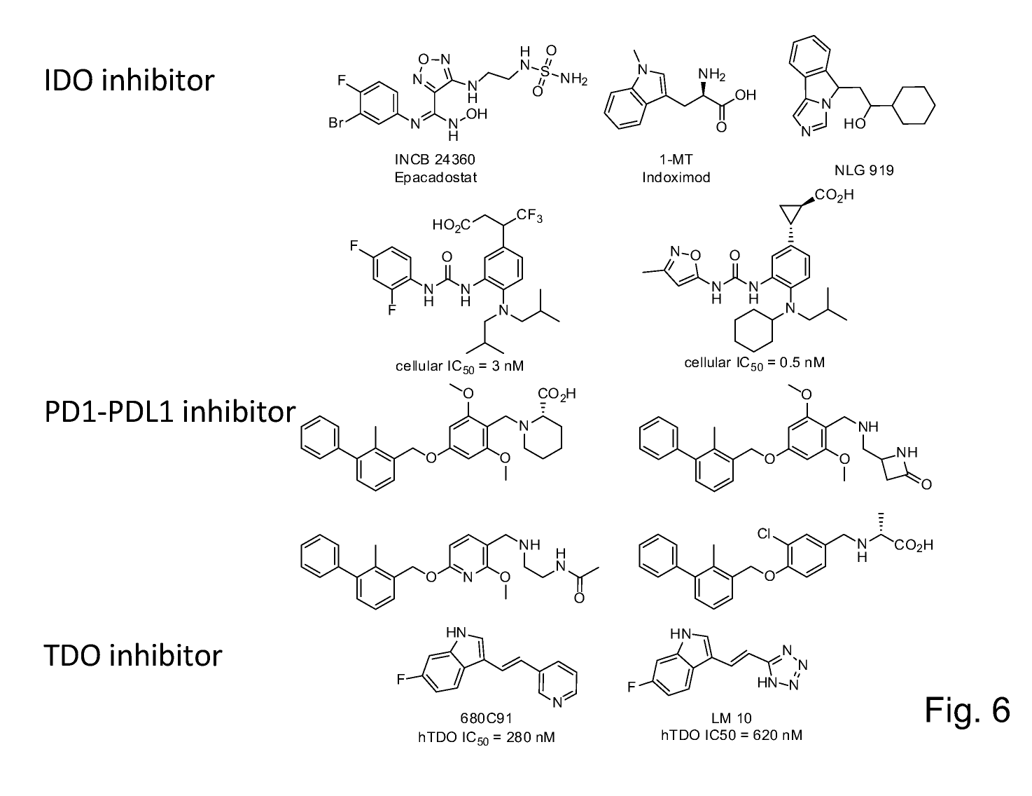

Although many of the studies hereof are focused on PTX, the systems, methods and compositions hereof may be readily extended to immunochemotherapy with other anticancer agents such as Dox etc. Further, the strategies hereof readily be employed to achieve immunochemotherapy that is targeted at other immune checkpoints such as PD-1. Representative examples of the chemical structure of a number of IDO inhibitors, PD1-PDL1 inhibitors, and TDO inhibitors suitable for user herein are set forth in FIG. 6.

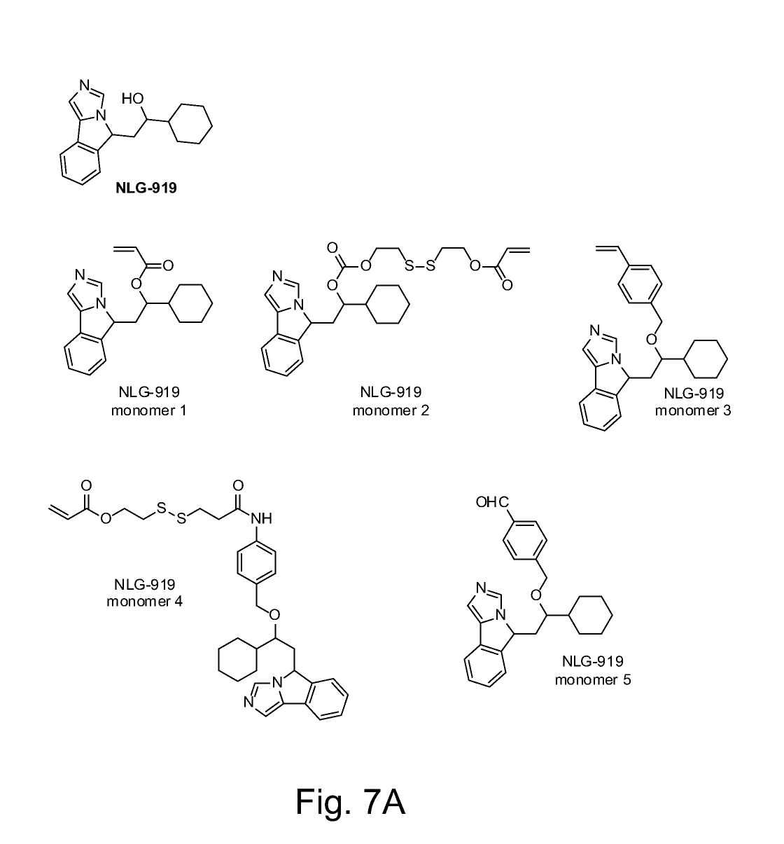

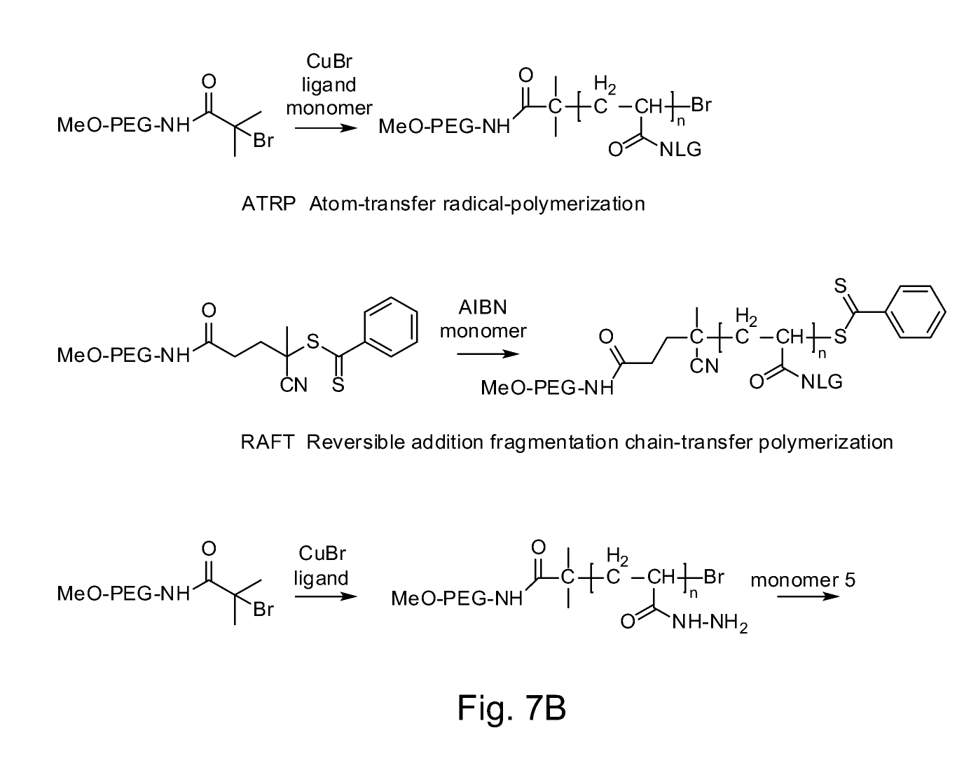

In a number of embodiments, immunotherapy agents incorporated into carrier agents hereof may be polymerized. For example, FIG. 7A illustrates NLG-919 and a number of polymerizable NLG-919 analogs or derivative monomers. Polymerizable NLG-919 analogs/monomers 1-4 of FIG. 7A include a double bond which can be polymerized via radical polymerization. NLG-919 analog/monomer 5 includes an aldehyde group that can react with hydrazine to form a pH sensitive bond (hydrazone). FIG. 7B illustrates two representative methods for polymerization. In the representative embodiments of FIG. 7B, a controlled/living radical polymerizations such as atom-transfer radical polymerization (ATRP) and reversible addition fragmentation chain transfer polymerization (RAFT) are illustrated.

Controlled/living polymerization is generally considered in the art to be a form of chain polymerization in which irreversible chain termination is substantially absent. An important feature of living polymerization is that polymer chains will continue to grow while monomer and reaction conditions to support polymerization are provided. Polymer chains prepared by living polymerization can advantageously exhibit a well-defined molecular architecture, a predetermined molecular weight and narrow molecular weight distribution or low polydispersity. Examples of living polymerization include ionic polymerization and controlled radical polymerization (CRP) in which termination cannot be completely avoided but can be strongly suppressed, in comparison with conventional radical polymerization. Examples of CRP include, but are not limited to, iniferter polymerization, stable free radical mediated polymerization (SFRP), atom transfer radical polymerization (ATRP), and reversible addition fragmentation chain transfer (RAFT) polymerization.

ATRP is considered to be one of the most successful controlled radical polymerization processes with significant commercial potential for production of many types materials. The process, including suitable transition metals and state of the art ligands, range of polymerizable monomers and materials prepared by the process, has been described in U.S. Pat. Nos. 5,763,548; 5,807,937; 5,789,487; 5,945,491; 6,111,022; 6,121,371; 6,124,411; 6,162,882; 6,407,187; 6,512,060; 6,538,091; 6,541,580; 6,624,262; 6,624,263; 6,627,314; 6,759,491; 6,790,919; 6,887,962; 7,019,082; 7,049,373; 7,064,166; 7,125,938; 7,157,530; 7,332,550; 7,572,874; 7,678,869; 7,795,355; 7,825,199; 7,893,173; 7,893,174, 8,252,880, 8,273,823; and 8,349,410, all of which are herein incorporated by reference. ATRP has also been discussed in numerous publications with Matyjaszewski as co-author and reviewed in several book chapters including Chem. Rev. 2001, 101, 2921-2990; Chem Rev 2007, 107, 2270-2299 and Prog. Polym. Sci., 2007, 32, 93-146, the disclosures of which are incorporated herein by reference.

Polymerization techniques other than CRP can also be used. As described above, for example, the aldehyde group of analog/monomer 4 of FIG. 7A can react with hydrazine to form hydrazine, which is a pH sensitive bond.

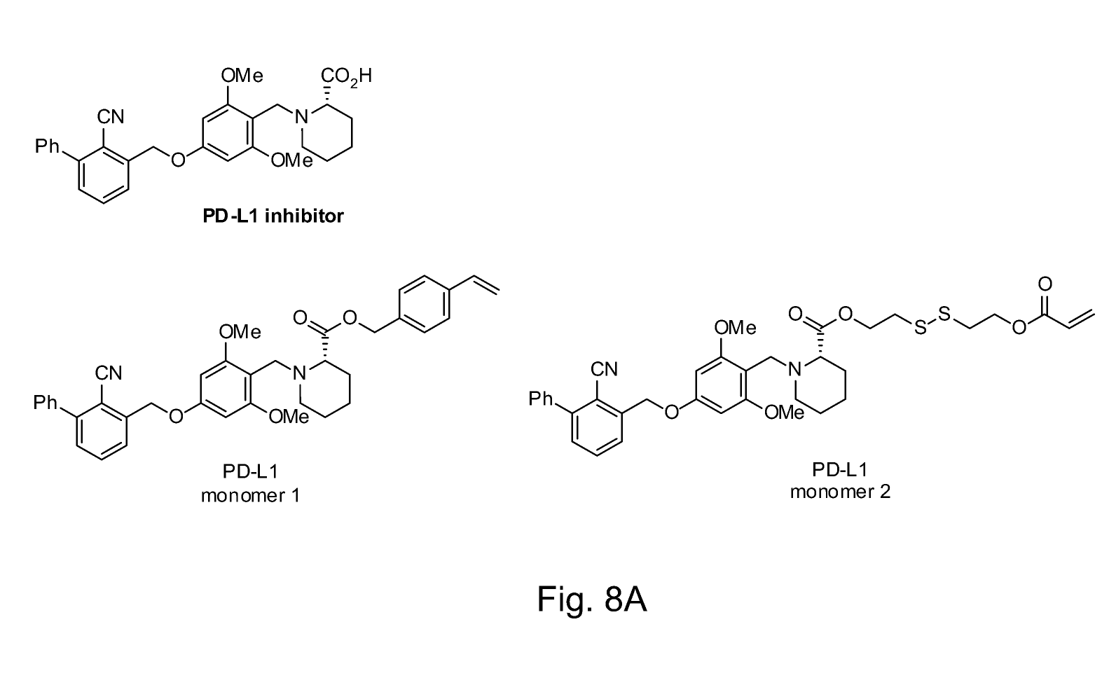

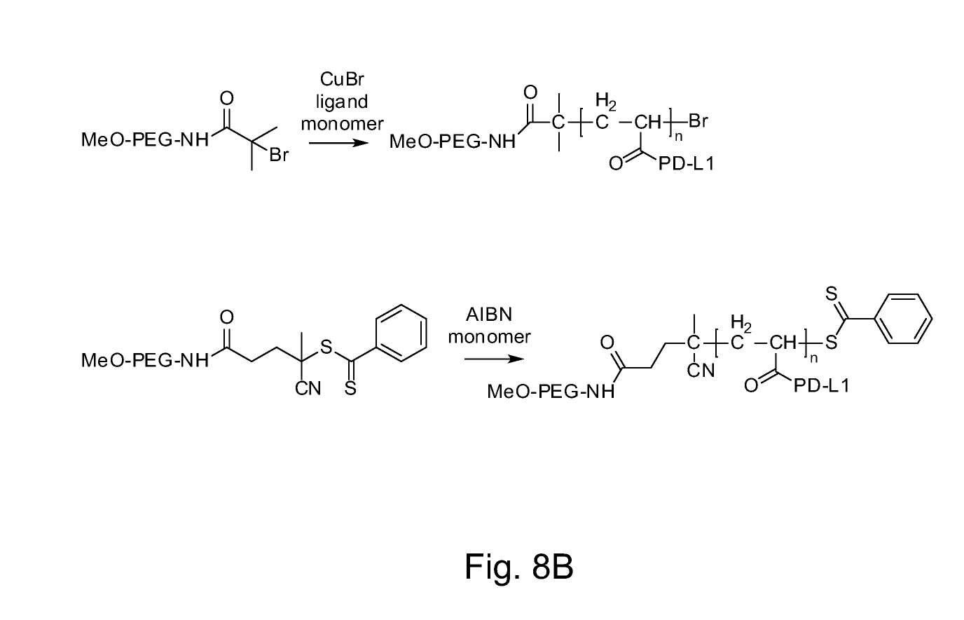

FIG. 8A illustrates a representative example of a PD-L1 inhibiting immunotherapy agent suitable for use herein and a number of polymerizable analogs/monomers thereof. FIG. 8B illustrates representative ATRP and RAFT polymerizations suitable for use with the analogs/monomers of FIG. 8A.

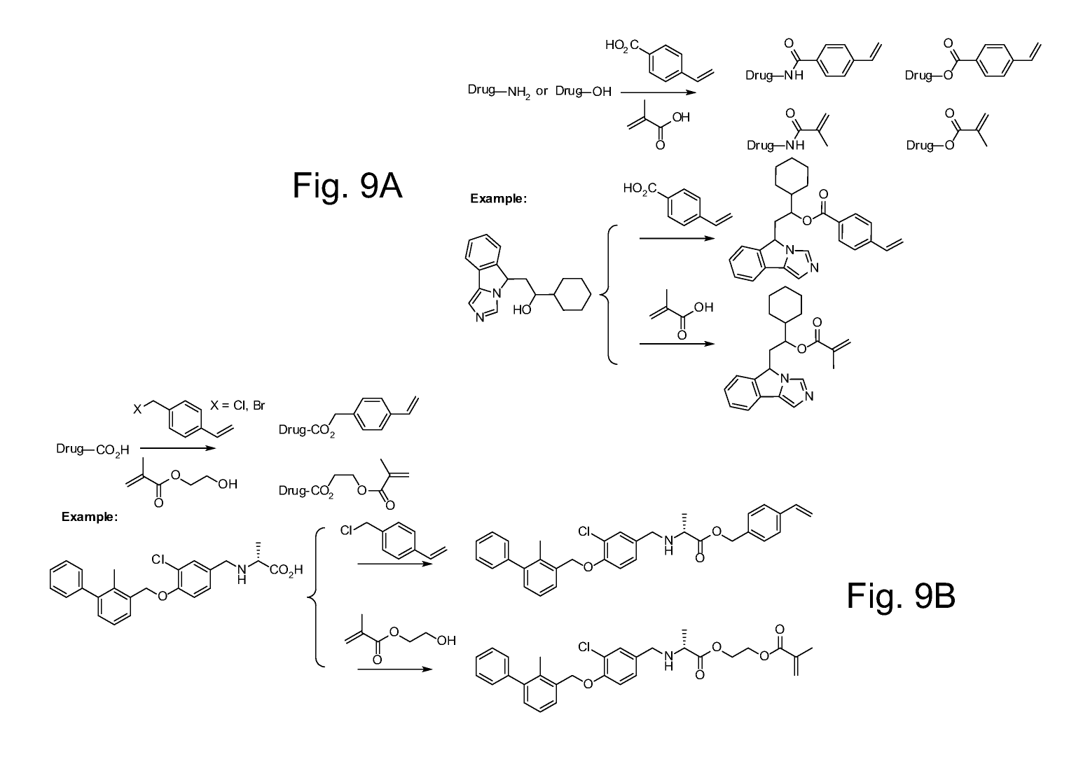

FIG. 9A sets forth generalized synthetic schemes for synthesis of monomers from drugs or agents including amino groups. FIG. 9B sets forth generalized synthetic schemes for synthesis of monomers from drugs or agents including carboxyl groups.

Experimental

Reagents. Paclitaxel (PTX, >99%) was purchased from TSZ Chem (MA, USA). Docetaxel (DTX, >99%) was obtained from LC Laboratories (MA, USA). α-Fmoc-ε-Boc-lysine, N, N′-dicyclohexylcarbodiimide (DCC), trifluoroacetic acid (TFA), and triethylamine (TEA) were purchased from Acros Organic (NJ, USA). Monomethoxy PEG₂₀₀₀, 4-dimethylaminopyridine (DMAP), ninhydrin, and other unspecified chemicals were all purchased from Sigma Aldrich (MO, USA). Dulbecco's phosphate buffered saline (DPBS), Dulbecco's Modified Eagle's Medium (DMEM), fetal bovine serum (FBS), penicillin-streptomycin solution (100×) were all purchased from Invitrogen (NY, USA). All solvents used in this study were HPLC grade.

Animal Studies. Female BALB/c mice (4-6 weeks), female BALB/c nude mice (4-6 weeks) and C57BL/6 mice (4-6 weeks) were purchased from Charles River (Davis, Calif.). All animals were housed under pathogen-free conditions according to AAALAC (Association for Assessment and Accreditation of Laboratory Animal Care) guidelines. All animal-related experiments were performed in full compliance with institutional guidelines and approved by the Animal Use and Care Administrative Advisory Committee at the University of Pittsburgh.

Cell cultures. 4T1.2 murine breast cancer cells, B16 murine melanoma cells, Panc02 murine pancreatic ductal adenocarcinoma cells, HeLa human cervical cancer cells, and PC3 human prostate cancer cells were maintained in Dulbecco's Modified Eagle's Medium (DMEM) supplemented with 10% fetal bovine serum (FBS) and 1% penicillin-streptomycin at 37° C. in a humidified environment with 5% CO₂.

Synthesis of PEG₂ₖ-Fmoc-NLG conjugate. Both PEG₂ₖ-Fmoc-NLG(L) and PEG₂ₖ-Fmoc-NLG(S) conjugates were synthesized by coupling NLG919 to PEG₂ₖ with either an ester or ether linkage. PEG₂ₖ-Fmoc-NLG(L) was synthesized as follow: 1 equiv. of monomethoxy PEG2000 was mixed with 3 equiv. of α-Fmoc-ε-Boc-lysine and DCC in dichloromethane (DCM) in the presence of DMAP for 2 days at room temperature (RT). Purified PEG_{2K}-Fmoc-lysine-Boc was obtained by filtering the mixture and then precipitation with ice-cold ether/ethanol twice. The Boc group was removed by treatment with DCM/TFA (1:1, v/v) for 2 hours at RT and the deprotected PEG_{2K}-lysine(Fmoc)-NH₂ was obtained by precipitation with ice-cold ether/ethanol. Finally, PEG₂ₖ-Fmoc-NLG(L) was synthesized by mixing PEG₂ₖ-lysine(Fmoc)-NH₂ with excess amount of NLG919, DCC, and small amount of DMAP in DCM at RT for 2 days. The mixture was filtered and the filtrate was precipitated by ice-cold ether/ethanol twice. The crude product was dissolved in water and filtered through a 450 nm filter, followed by lyophilization to yield the powder of purified PEG₂ₖ-Fmoc-NLG(L). To synthesize PEG₂ₖ-Fmoc-NLG(S), NLG919 was reacted with methyl 4-bromobutanoate to form ether bond under NaH condition. After column purification, the methyl ester was hydrolyzed by NaOH and the obtained compound (3 equiv.) was conjugated with PEG₂ₖ-lys(Fmoc)-NH₂ (1 equiv.) using DCC (3 equiv.) and DMAP (0.3 equiv.). The mixture was filtered and the clear filtrate was precipitated by ice-cold ether/ethanol twice. The crude product was dissolved in water, filtered, and lyophilized to obtain the purified PEG₂ₖ-Fmoc-NLG(S).

Cell-based IDO assays. The IDO inhibitory effect of PEG₂ₖ-Fmoc-NLG was tested by an in vitro IDO assay See, for example, Liu X, et al. Selective inhibition of IDO1 effectively regulates mediators of antitumor immunity. Blood 115, 3520-3530 (2010). Briefly, HeLa cells were seeded in a 96-well plate at a cell density of 5×10³ cells per well and allowed to grow overnight. Recombinant human IFN-γ was then added to each well with a final concentration of 50 ng/mL. At the same time, various concentrations of PEG₂ₖ-Fmoc-NLG(L), PEG₂ₖ-Fmoc-NLG(S) or free NLG919 (NLG919 concentrations: 50 nM-20 μM) were added to the cells. After 48 h of incubation, 150 μL of the supernatants per well was transferred to a new 96-well plate. Seventy-five μl of 30% trichloroacetic acid was added into each well and the mixture was incubated at 50° C. for 30 min to hydrolyze N-formylkynurenine to kynurenine. For colorimetric assay, supernatants were transferred to a new 96-well plate, mixed with equal volume of Ehrlich reagent (2% p-dimethylamino-benzaldehyde w/v in glacial acetic acid), and incubated for 10 min at RT. Reaction product was measured at 490 nm by a plate reader. For HPLC-MS/MS detection (Wastes Alliance 2695 Separation Module combined with Waters Micromass Quattro Micro TM API MS detector), the plate was centrifuged for 10 min at 2500 rpm and 100 μl of the supernatants per well was collected for tryptophan and kynurenine assay.

T cell proliferation study. A lymphocyte-Panc02 cell co-culture study was conducted to examine whether PEG₂ₖ-Fmoc-NLG can reverse IDO1-mediated inhibition of T cell proliferation^{21, 25}. Murine Panc02 cells were stimulated by IFN-γ (50 ng/ml) to induce IDO expression and then irradiated (6000 rad) before coculture. Splenocyte suspensions were generated from BALB/c mice by passage through the nylon wool columns after lysing of red blood cells. IFN-γ-stimulated Panc02 cells (1×10⁵ cell/well) were mixed with splenocytes (5×10⁵ cells per well, pre-stained with CSFE) in a 96 well plate. Various concentrations of NLG919, PEG₂ₖ-Fmoc-NLG(L) or PEG₂ₖ-Fmoc-NLG(S) were added to the cells. To measure the T cell proliferation, 100 ng/mL anti-CD3 and 10 ng/mL mouse recombinant IL-2 were added to the cocultures. The proliferation of CD8⁺ and CD4⁺T cells was measured by FACS after 3 days of coculture.

Measurements of Trp and Kyn in plasma and tumor tissues. The kynurenine to tryptophan ratios in plasma or tumors in 4T1.2 tumor-bearing mice following different treatments were examined by LC-MS/MS as an indication of IDO enzyme activity³². BALB/c mice bearing 4T1.2 tumors of ˜50 mm³ were treated with DPBS, TAXOL (10 mg PTX/kg), PEG₂ₖ-Fmoc-NLG(L), or PTX/PEG₂ₖ-Fmoc-NLG(L) (10 mg PTX/kg) via tail vein once every 3 days for 5 times. One day after the last treatment, the plasma and tumor samples were harvested. Plasma samples were mixed with methanol (plasma: methanol, 1:2.5, v/v) and centrifuged at 14,500 rpm for 15 min. Supernatants were collected for LC-MS quantification of kynurenine and tryptophan.

Tumor samples were homogenized in water and the homogenates were mixed with acetonitrile (1:1, v/v), centrifuged and supernatants were transferred to clean tubes. Equal volumes of methanol were added to precipitate proteins and supernatants were collected following centrifugation for HPLC-MS/MS measurement.

Quantification of tumor-infiltrating lymphocytes. BALB/c mice bearing 4T1.2 tumors of ˜50 mm³ received various treatments via tail vein injection once every 2 days for 5 times. Tumors and spleen were harvested one day following the last treatment. Single cell suspensions were prepared and costained for CD4, CD8, IFN-γ, Granzyme B, Foxp3, myeloid-derived suppressor cell (CD11b and Gr-1) and macrophage (F4/80 and CD206) for FACS analysis.

In vivo therapeutic study of PEG₂ₖ-Fmoc-NLG micelles alone in a murine breast cancer model (4T1.2 ). To investigate whether IDOL inhibition by PEG₂ₖ-Fmoc-NLG micelles can suppress tumor growth, female BALB/c mice of 4-6 weeks old were s.c. inoculated with 4T1.2 tumor cells (2×10⁵ cells/mouse)^{20, 33} Mice were randomly grouped (N=5) when the tumor volume reached ˜50 mm³ and treated with PEG₂ₖ-Fmoc-NLG(L), PEG₂ₖ-Fmoc-NLG919(S), or NLG formulated in PEGSk-(Fmoc-Boc)₂ micelles (25 mg NLG919/kg) once every 3 days for 5 times via tail vein injection. A separate group was treated with NLG919 orally once daily for 15 days. Tumor sizes were measured twice weekly in two dimensions using a caliper, and the tumor volumes were calculated with the formula: V=(A×B²)/2 (A and B are the long and short diameters of the tumor). Relative tumor volume was calculated to compare different treatment groups (relative tumor volume=tumor volume/tumor volume prior to first treatment). Mice were sacrificed when tumor volume reached ˜2000 mm³. The difference between different treatment groups was analyzed by ANOVA with significance defined as P<0.05. The above study was similarly performed in BALB/c nude mice to elucidate a role of T cell response in PEG₂ₖ-Fmoc-NLG-mediated antitumor activity. See, for example, Liu X, et al. Selective inhibition of IDO1 effectively regulates mediators of antitumor immunity. Blood 115, 3520-3530 (2010); and Hou D Y, et al. Inhibition of indoleamine 2,3-dioxygenase in dendritic cells by stereoisomers of 1-methyl-tryptophan correlates with antitumor responses. Cancer research 67, 792-801 (2007).

Preparation and characterizations of drug-free or drug-loaded PEG₂ₖ-Fmoc-NLG micelles. The drug-loaded micelles were prepared by mixing PTX (10 mM in chloroform) or DOX (10 mM in chloroform) with PEG₂ₖ-Fmoc-NLG(L) or PEG₂ₖ-Fmoc-NLG(S) (10 mM in chloroform) at various carrier/drug ratios. The solvent was removed by N₂ flow to form a thin film of drug/carrier mixture. The film was dried under vacuum for 1 h and DPBS was added to form the drug-loaded micelles. The particle size and zeta potential of micelles were measured by a Zetasizer. The morphologies of both drug-free micelles and drug-loaded micelles were examined by transmission electron microscopy (TEM). The critical micelle concentration (CMC) was determined by using nile red as a fluorescence probe following the protocol set forth in Handke N, et al. Elaboration of glycopolymer-functionalized micelles from an N-vinylpyrrolidone/lactide-based reactive copolymer platform. Macromolecular bioscience 13, 1213-1220 (2013).

In vitro cytotoxicity of PTX- and DOX-loaded PEG₂ₖ-Fmoc-NLG(L) micelles. 4T1.2 , PC3, Panc02, and B16 cells at 2000 cells/well were seeded in 96-well plates, respectively. After 12 h incubation, the cell culture medium was removed and various concentrations of free PTX, free PEG₂ₖ-Fmoc-NLG(L) micelles or PTX/PEG₂ₖ-Fmoc-NLG(L) mixed micelles were added to the cells. After 3 days of incubation, 20 μL of 3-(4,5-dimethylthiazol-2-yl)-2,5-diphenyltetrazoliumbromide (MTT) in DPBS (5 mg/mL) was added to each well and cells were further incubated for 2 h. Medium was removed and MTT formazan crystals were solubilized by 100 μL of DMSO per well. Absorbance of each well was measured with a microplate reader at wavelength of 550 nm. Untreated wells were used as controls. Cell viability was calculated as [(OD_{treated}-OD_{blank})/(OD_{control}-OD_{blank})×100%]. Cytotoxicity of DOX-loaded PEG₂ₖ-Fmoc-NLG(L) micelles was similarly examined.

Plasma pharmacokinetics and tissue distribution. Groups of 5 female BALB/c mice were i.v. administered with TAXOL or PTX/PEG₂ₖ-Fmoc-NLG(L) mixed micelles at a dose of 10 mg PTX/kg. Blood samples of 50 μL were withdrawn from the retro-orbital plexus/sinus of the mice from 3 min to 12 h (3 min, 10 min, 30 min, 1 h, 2 h, 4 h, 8 h, and 12 h). The blood collected in heparinized tubes was centrifuged at 2,500 rpm for 15 min. To 20 μL of plasma, 350 μL of acetonitrile was added for protein precipitation and the resulting mixture was centrifuged at 12,000 rpm for 5 min. Three-hundred microliters of the supernatants were collected from each sample and dried under airflow. The residues were dissolved in 50 μL of methanol and analyzed by HPLC for PTX. The pharmacokinetic parameters were calculated based on a noncompartment model by Phoenix WinNonlin.

For tissue distribution study, groups of 5 BALB/c mice bearing 4T1.2 tumors of 400-600 mm³ were i.v. administered with PTX-loaded PEG₂ₖ-Fmoc-NLG (L) micelles or TAXOL at a PTX dose of 10 mg/kg. Mice were sacrificed 24 h after injection. Major organs and tumor tissues were collected, weighed, and homogenized with 2 mL solvent (acetonitrile to H₂O=1:1, v/v). The samples were centrifuged at 4° C., 3,500 rpm for 15 min, and the supernatants were collected and dried under airflow. The residues were then dissolved in 200 μL solvent (Methanol to H₂O=1:1, v/v) and centrifuged at 4° C., 14,500 rpm for 10 min. The supernatants were mixed with equal volume of methanol and centrifuged again at 4° C., 14,500 rpm for 10 min. Twenty microliters of the clear supernatants were injected into HPLC system for detection of PTX.

The kinetics and biodistribution of PEG₂ₖ-Fmoc-NLG (L) and NLG919-loaded PEG₅ₖ-(Fmoc-Boc)₂ micelles were similarly performed as described above. Both released free NLG and total NLG (free NLG plus intact PEG₂ₖ-Fmoc-NLG (L)) were determined. Briefly, following the extraction from the blood or tissues, samples were treated with porcine liver esterases (Sigma) at a final concentration of 50 U/mL. After 48 h, the total NLG (released free NLG plus NLG cleaved from PEG₂ₖ-Fmoc-NLG by the added esterases) was extracted twice by dichloromethane (2×2 ml) and dried under airflow. The samples were then similarly processed as described above and determined by a LC-MS system (Wastes Alliance 2695 Separation Module combined with Waters Micromass Quattro Micro TM API MS detector).

In vivo antitumor activity of PTX/PEG₂ₖ-Fmoc-NLG(L). In vivo antitumor activity of PTX formulated in PEG₂ₖ-Fmoc-NLG(L) micelles was similarly examined in 4T1.2 tumor model as described above. Controls included PEG₂ₖ-Fmoc-NLG(L), TAXOL, PTX/PEG₂ₖ-Fmoc-NLG(S), (PTX+NLG)/PEG₅ₖ-(Fmoc-Boc)₂ , oral NLG plus i.v. Abraxane, and PEG₂ₖ-Fmoc-NLG(L) plus Abraxane. The PTX dose was 10 mg/kg and mice received all i.v. treatments once every 3 days for 5 times. Oral NLG was given daily for 15 days. The growth of tumors was followed every three days after initiation of treatment for 19 days and relative tumor volume was calculated. The difference between different treatment groups was analyzed by ANOVA with significance defined as P<0.05. The tumors were harvested and weighted at the end of experiment.

Similarly, a dose escalation study (5, 10, and 20 mg PTX/kg) was conducted for PTX/PEG₂ₖ-Fmoc-NLG(L) in 4T1.2 tumor model. The antitumor activity of PTX/PEG₂ₖ-Fmoc-NLG(L) was further examined in a murine melanoma model, B16, as described above.

The immune cell populations in the tumor tissues with various treatments were analyzed by flow cytometry. See, for example, Broz M L, et al. Dissecting the tumor myeloid compartment reveals rare activating antigen-presenting cells critical for T cell immunity. Cancer Cell, 26, 638-652 (2014)

Statistics All data are presented as mean±s.e.m. Differences between groups were assessed using ANOVA and P<0.05 was considered statistically significant.

The foregoing description and accompanying drawings set forth a number of representative embodiments at the present time. Various modifications, additions and alternative designs will, of course, become apparent to those skilled in the art in light of the foregoing teachings without departing from the scope hereof, which is indicated by the following claims rather than by the foregoing description. All changes and variations that fall within the meaning and range of equivalency of the claims are to be embraced within their scope.

1. A formulation, comprising:.
a carrier agent formed by conjugating an immunotherapy agent with a hydrophilic compound, the carrier agent further comprising an interactive domain comprising at least one interactive moiety which interacts with a therapeutic agent.

2. The formulation of claim 1 wherein the immunotherapy agent is conjugated to the hydrophilic compound via a linkage which is labile in vivo.

3. The formulation of claim 2 wherein the at least one interactive moiety interacts with a chemotherapy agent.

4. The formulation of claim 2 wherein the immunotherapy agent affects programmed cell death protein, indoleamine-pyrrole 2,3-dioxygenase, cytotoxic T-lymphocyte antigen 4(CTLA-4), PD-L1, PD-L2, lymphocyte activation gene 3(LAG3), or B7 homolog3(B7-H3)

5. The formulation of claim 2 wherein the interactive domain comprises at least one of a fluorenylmethyloxycarbonyl group, a carbobenzyloxy group, an isobutoxycarbamate group, a naphthylacetyl group, a carbazole group, a quinolone group, an isoquinolone group, or a group which is a residue of a molecule selected from the group of the therapeutic agent, a portion of the therapeutic agent, (9H-fluoren-9-yl)methanamine, (9H-fluoren-9-yl)methanol, 9H-fluoren-9-amine, naphthalene, 1,1′-bi-2-naphthol (BINOL), camptothecin, a camptothecin analog, pemetrexed, docetaxel, paclitaxel, epirubicin, doxorubicin, vinblastine, vindesine, etoposide, hydroxycamptothecin, irinotecan, mitoxantrone, tamoxifen, tretinoin, Vitamin A, Vitamin E, Vitamin K, Vitamin D, curcumin, imatinib, gefitinib, erlotinib, sorafenib, and bortezomib, or a derivative thereof.

6. The formulation of claim 2 wherein the interactive domain comprises at least one fluorenylmethyloxycarbonyl group or a derivative thereof.

7. The formulation of claim 2 wherein the hydrophilic compound comprises at least one hydrophilic oligomer or at least one hydrophilic polymer.

8. The formulation of claim 7 wherein the hydrophilic oligomer or the hydrophilic polymer is a polyalkylene oxide, a polyvinylalcohol, a polyacrylic acid, a polyacrylamide, a polyoxazoline, or a polypeptide.

9. The formulation of claim 8 wherein the polyalkylene oxide is a polyethylene glycol.

10. The formulation of claim 2 wherein the at least one interactive moiety has an affinity for the therapeutic agent.

11. The formulation of claim 10 wherein the at least one interactive moiety interacts with the therapeutic agent via π-π stacking, hydrophobic interaction or hydrogen-bonding.

12. The formulation of claim 2 wherein the carrier agent provides a loading capacity for the therapeutic agent of at least 10%.

13.-14. (canceled)

15. The formulation of claim 2 wherein the immunotherapy agent is NLG919 or derivative thereof.

16. The formulation of claim 15 wherein the interactive domain comprises at least one of a fluorenylmethyloxycarbonyl group, a carbobenzyloxy group, an isobutoxycarbamate group, a naphthylacetyl group, a carbazole group, a quinolone group, an isoquinolone group, or a group which is a residue of a molecule selected from the group of the therapeutic agent, a portion of the therapeutic agent, (9H-fluoren-9-yl)methanamine, (9H-fluoren-9-yl)methanol, 9H-fluoren-9-amine, naphthalene, 1,1′-bi-2-naphthol (BINOL), camptothecin, a camptothecin analog, pemetrexed, docetaxel, paclitaxel, epirubicin, doxorubicin, vinblastine, vindesine, etoposide, hydroxycamptothecin, irinotecan, mitoxantrone, tamoxifen, tretinoin, Vitamin A, Vitamin E, Vitamin K, Vitamin D, curcumin, imatinib, gefitinib, erlotinib, sorafenib, and bortezomib, or a derivative thereof.

17. The formulation of claim 16 wherein the interactive domain comprises at least one fluorenylmethyloxycarbonyl group or a derivative thereof.

18. The formulation of claim 17 wherein the hydrophilic compound comprises at least one hydrophilic oligomer or at least one hydrophilic polymer.

19.-21. (canceled)

22. The formulation of claim 3 wherein the chemotherapeutic agent is paclitaxel, doxorubicin, docetaxel, gefitinib, imatinib, dasatinib, curcumin, camptothecin, etoposide, edelfosine, vincristine, temsirolimus, carmustine or a chemotherapeutically active derivative thereof.

23. The formulation of claim 2 further comprising the therapeutic agent.

24. The formulation of claim 23 wherein the therapeutic agent is paclitaxel, doxorubicin, docetaxel, gefitinib, imatinib, dasatinib, curcumin, camptothecin, etoposide, edelfosine, vincristine, temsirolimus, carmustine or a chemotherapeutically active derivative thereof.

25. The formulation of claim 2 wherein the immunotherapeutic agent is a polymer formed from immunotherapeutically active monomers.

26. A method of forming a formulation, comprising:.
forming a carrier agent by conjugating an immunotherapy agent with a hydrophilic compound, the carrier agent further comprising an interactive domain comprising at least one interactive moiety which interacts with a therapeutic agent.

27. The method of claim 26 wherein at least one interactive group is selected to interact with a chemotherapy agent.

28. (canceled)

29. The method of claim 27 further comprising adding the therapeutic agent to the carrier agent to complex the chemotherapy agent to the carrier agent.

30. The method of claim 29 wherein the immunotherapy agent is conjugated to the hydrophilic compound via linkage which is labile in vivo.

31. A method of treating a patient with a therapeutic agent, comprising: delivering to the patient a formulation, wherein the formulation comprises the therapeutic agent and a carrier agent formed by conjugating an immunotherapy agent with a hydrophilic compound, the carrier agent further comprising an interactive domain comprising at least one interactive moiety which interacts with the therapeutic agent.

32. The method of claim 31 wherein the interactive domain is positioned between a residue of the therapeutic agent and a residue of the hydrophilic compound in the carrier agent.

33. The method of claim 32 wherein the immunotherapy agent is conjugated to the hydrophilic compound via a linkage which is labile in vivo.
